(19) United States
(12) Patent Application Publication
(10) Pub. No.: US 2023/0149307 A1
(43) Pub. Date: May 18, 2023

(54) POROUS DOUBLE-NETWORK HYDROGEL

(71) Applicant: THE ROYAL INSTITUTION FOR THE ADVANCEMENT OF LEARNING/MCGILL UNIVERSITY, Montréal (CA)

(72) Inventors: Guangyu BAO, Montréal (CA); Jianyu LI, Verdun (CA); Luc MONGEAU, Mont Royal (CA); Sareh TAHERI, Montréal (CA); Sepideh MOHAMMADI, Montréal (CA)

(21) Appl. No.: 18/054,292

(22) Filed: Nov. 10, 2022

Related U.S. Application Data
(60) Provisional application No. 63/278,288, filed on Nov. 11, 2021.

(51) Int. Cl. A61K 9/06; C08J 3/075; A61K 47/36; A61K 47/32; A61P 7/04; A61P 17/02; A61K 35/36; A61K 9/00

(52) U.S. Cl. CPC A61K 9/06; C08J 3/075; A61K 47/36; A61K 47/32; A61P 7/04; A61P 17/02; A61K 35/36; A61K 9/0019; C08J 2305/08; C08J 2333/26

(57) ABSTRACT

There is provided a porous double-network hydrogel comprising: a first network comprising a first polymer; a second network comprising a second polymer. The porous double-network hydrogel comprises pores having a diameter of at least 1 μm, and the porous double-network hydrogel is perfusable and injectable.

### CROSS REFERENCE TO A RELATED APPLICATION

The present application claims priority to U.S. Provisional Application No. 63/278,288 filed on Nov. 11, 2021, which is incorporated herein by reference in its entirety.

### STATEMENT OF GOVERNMENT RIGHTS

This work was funded at least in part by a United States Grant from the National Institutes of Health. The government may have certain rights to this invention.

### TECHNICAL FIELD

This disclosure relates to the field of porous double-network hydrogels (PDN), methods and uses thereof and methods of fabricating same.

### BACKGROUND OF THE ART

Injectable hydrogels can be delivered via needle-syringe injection into the human body with limited invasiveness. Injectable hydrogels have been used in many branches of medicine, including drug/cell delivery, tissue engineering, biofabrication, organs-on-chips, and disease modeling. However, there still remain challenges concerning the mass transport and mechanical properties of injectable hydrogels. On one hand, most injectable hydrogels in use or under development are not perfusable due to their nanoporous structures. This issue limits the rapid transport of oxygen and nutrients (diffusion depth ˜600 μm), as well as the trafficking of native or transplanted cells. The adverse effects can also be found in microfluidics, such as organs-on-chips. Direct injection of hydrogels into microchannels tends to block the perfusion channel and thereby disable the devices. Immediate vascularization is therefore desired but difficult to realize through injection. On the other hand, many injectable hydrogels cannot sustain large deformations and are susceptible to fracture in mechanically dynamic tissue environments. An extreme case is the vocal folds, a mechanically dynamic organ in the human body. Implants in the vocal fold lamina propria are exposed to up to 50% strains at a fundamental frequency that is on the order of 10² Hz. Currently, patients with vocal fold injuries suffer from repeated hydrogel injections, due in part to the fracture-induced short lifetime of the hydrogel implants under the dynamic loadings. By contrast, many biological tissues are perfusable and yet tough to tolerate extreme biomechanical stimulations as part of their normal functions, as exemplified by the vocal fold and heart. To close the gap between injectable hydrogels and biological tissues, strategies to achieve a combination of high permeability and mechanical toughness are highly desired.

The incorporation of porous structures with injectable hydrogels has been realized to enable medium perfusion in lieu of vascularization. Those porous structures can be preformed before injection or formed in-situ after placement in the human body. Preformed porous hydrogels can collapse to pass through a needle, and then regain their shape post-injection. To prevent damage to such hydrogels during injection, the use of oversized needles is necessary but increases the invasiveness of the procedure. In addition, the preformed method usually demands lengthy fabrication processes, such as cryogelation, lyophilization, or 3D printing, as well as prior knowledge about the shape and volume of the injection site to conform to the often irregular wound. Alternatively, in-situ pore-forming hydrogels are preferable as they can be delivered in liquid form through small-sized needles and undergo a sol-gel transition into porous scaffolds in the human body. Such hydrogels have been developed using either porogen, leachable particles, nanoclay, granular particles, self-assembly, or polymer degradation. Bioprintable pore-forming formulas, such as aqueous two-phase emulsion system and hydrogel microstrands, could be used for injection as well. These single network hydrogels, however, suffer from limited pore size (e.g. 0.1-100 μm). Increasing the pore size of these single network hydrogels will decrease their interconnectivity, which impairs their permeability, mechanical properties and performance. Attempts to promote perfusion and interconnectivity by enlarging the pores deteriorated their mechanical strength because pores essentially act as defects or cracks. This issue is more prevalent when the hydrogels are subjected to the biomechanical stimulations present in mechanically dynamic organs or tissues. Examples of single network hydrogels include alginate hydrogels which have pore size of 5-17 nm and a porosity of ˜0% (August et al. 2006); agarose hydrogels which have pore size of 8-40 nm, a porosity of ˜0%, and permeability of 10⁻¹⁷−10⁻¹⁶ m² (August et al. 2006); polyacrylamide hydrogels which have pore size of ˜10 nm, a porosity of ˜0%, and a permeability of 10⁻¹⁸−10⁻¹⁶ m² (Kapur et al. 1996); polyethylene glycol-based hydrogels which have pore size of 7-25 nm, a porosity of ˜0%, and a permeability of 10⁻¹⁷−10⁻¹⁵ m² (Offedu et al. 2018); and gelatin hydrogels which have pore size of 12-30 nm, a porosity of ˜0%, and a permeability of 10⁻¹⁸−10⁻¹⁵ m² (Koshy et al.).

Circumventing the inverse correlation between porosity and toughness while ensuring injectability and cytocompatibility has proven to be a challenge. Recently, a variety of double-network (DN) hydrogels have been reported to tolerate large defects and pores thanks to their high fracture toughness (X. Zhao et al., 2021). The pore-insensitivity is attributed to the synergy of dissipative and stretchy networks, which are often denoted as primary and secondary networks, respectively. These strategies, however, cannot meet other above-mentioned requirements for injectable perfusable hydrogels. While the dissipative primary network can be realized with biopolymers at mild conditions, there are concerns about the employment of the stretchy secondary network, which requires toxic precursors and/or harsh reaction conditions. To render the hydrogel precursor cytocompatible, synthetic and naturally derived polymers have been used as the secondary network components, including clickable polyethylene glycol, methacrylated hyaluronic acid, 4-carboxyphenylboronic acid grafted poly(vinyl alcohol), and polyacrylamide-co-diacetone acrylamide. The resulting tough hydrogels are nonetheless nanoporous. Naturally derived fibrous networks, such as platelet-rich fibrin, have also been explored to form the secondary network but at the expense of significantly reduced stretchability. It is also noted that a high toughness often restricts activities for the cells that reside within and hinders their cellular functions. Accordingly, improvements in injectable hydrogels to achieve improved permeability and mechanical toughness are sought.

### SUMMARY

In one aspect there is provided a porous double-network hydrogel comprising a first network comprising a first polymer; a second network comprising a second polymer; and wherein the porous double-network hydrogel and comprises pores having a diameter of at least 1 μm, and wherein the porous double-network hydrogel is perfusable and injectable. In one embodiment, the pores have a maximal diameter of 100 μm. In one embodiment, the weight to volume ratio of the weight of the first polymer to the volume of the hydrogel is from 0.25 to 4%. In one embodiment, the weight to volume ratio of the weight of the second polymer to the volume of the hydrogel is from 0.15 to 8%. The porous double-network hydrogel can have a porosity of 18 to 70%. The porous double-network hydrogel can have a fracture toughness of at least 5 J m⁻². The porous double-network hydrogel can have a permeability of from 10⁻¹⁴ to 10⁻¹² m². The porous double-network hydrogel can have a half-life time of stress relaxation from 10 to 100 s. The porous double-network hydrogel can have a fractocohesive length of from 0.25 mm to 0.5 mm. In some embodiments, the first polymer has a pKa of from 6 to 6.5. In further embodiments, the first polymer is chitosan or poly(N-isopropylacrylamide). In still further embodiments, the second polymer is glycol-chitosan, polyacrylamide, collagen, fibrin, polyethylene glycol or gelatin. In yet further embodiments, the first polymer is physically self crosslinked. Additionally, in yet further embodiments, the second polymer is covalently crosslinked, which can be self crosslinked or enzymatically crosslinked (e.g. peptide bonds).

There is also provided the use of the double-network hydrogel according to the present disclosure in the treatment of a hemorrhage, in the treatment of an injury, and/or in cellular therapy for a subject in need thereof. There is further provided a method of treating a subject in need thereof by injecting the porous double-network hydrogel of the present disclosure to a hemorrhagic site and/or an injury site in the subject. The hemorrhagic site and/or the injury site may be the vocal fold. Finally, there is provided a cellular therapy method comprising injecting the porous double-network hydrogel of the present disclosure and therapeutic cells in a subject in need thereof.

Many further features and combinations thereof concerning the present improvements will appear to those skilled in the art following a reading of the instant disclosure.

### DESCRIPTION OF THE DRAWINGS

FIG. 1A is a schematic diagram of a method of using a porous double-network hydrogel (PDN) according to an embodiment of the present disclosure in a vocal fold graft.

FIG. 1B is a graph of the moduli as a function of time showing the thermal gelation kinetics of PDN when the temperature is raised from room temperature to 37° C. (G′ is the storage moduli and G″ the loss moduli).

FIG. 1C is a bar graph showing the storage modulus for nanoporous single-network (NSN), porous single-network (PSN), double-network hydrogel with 0.5 w/v percentage of glycol-chitosan (PDN_{0.5}), double-network hydrogel with 1 w/v percentage of glycol-chitosan (PDN₁), and double-network hydrogel with 1.5 w/v percentage of glycol-chitosan (**** represents P<0.0001, n.s. is P>0.05).

FIG. 1D is a bar graph showing the half-life time of stress relaxation (t_{1/2}) of NSN, PSN, and PDN_{0.5}, PDN₁ and PDN_{1.5} (** represents P<0.01 and ***P<0.001).

FIG. 1E is a confocal image of a hydrogel containing FITC-labeled chitosan and glycol-chitosan (NSN).

FIG. 1F is a confocal image of a hydrogel containing FITC-labeled chitosan and glycol-chitosan (PSN).

FIG. 1G is a confocal image of a hydrogel containing FITC-labeled chitosan and glycol-chitosan (PDN₁).

FIG. 1H is graph of the fluorescent signal distribution from the hydrogel of FIG. 1E.

FIG. 1I is graph of the fluorescent signal distribution from the hydrogel of FIG. 1E.

FIG. 1J is graph of the fluorescent signal distribution from the hydrogel of FIG. 1E

FIG. 1K is a bar graph showing the size of NSN, PSN, and PDN_{0.5}, PDN₁ and PDN_{1.5} (**** represents P<0.0001).

FIG. 1L is a bar graph showing the porosity of NSN, PSN, and PDN₀₅, PDN₁ and PDN_{1.5} (**** represents P<0.0001).

FIG. 2A is a schematic of a fabrication process and design for the phonomimetic bioreactor system.

FIG. 2B is a schematic of the configuration of the control loop and arrangement of the complete bioreactor setup.

FIG. 2C is a photographic configuration of the control loop and arrangement of the complete bioreactor setup.

FIG. 3 is a graph of the complex viscosity of PDN₁ as a function of time after mixing at room temperature. Black dots denote the mean value, and the grey area denotes the standard deviation.

FIG. 4 is a graph showing the moduli in function of time for the gelation kinetics of chitosan and glycol-chitosan.

FIG. 5 is a bar graph showing the Young's moduli of NSN, PSN, PDN_{0.5}, PDN₁ and PDN₂ calculated from compression tests (****P<0.0001, n.s. means P≥0.05).

FIG. 6 is a bar graph of the stress relaxation time of NSN, PSN, PDN_{0.5}, PDN₁ and PDN₂ evaluated by the stress retention 1/e.

FIG. 7A is a confocal image of NSN.

FIG. 7B is a confocal image of PSN.

FIG. 7C is a confocal image of PDN_{0.5}.

FIG. 7D is a confocal image of PDN₁.

FIG. 7E is a confocal image of PDN₂.

FIG. 7F is a scanning electron microscopy (SEM) image of NSN.

FIG. 7G is a SEM of PSN.

FIG. 7H is a SEM of PDN_{0.5}.

FIG. 7I is a SEM of PDN₁.

FIG. 7J is a SEM of PDN₂.

FIG. 7K is a side view microcomputed tomography (μCT) scan of NSN (prior art).

FIG. 7L is a close up of the μCT scan of FIG. 7K, specifically of the square delimitated on FIG. 7K.

FIG. 7M is a top view μCT scan of NSN (prior art).

FIG. 7N is a side view μCT scan of PDN₁.

FIG. 7O is a close up of the μCT scan of FIG. 7N, specifically of the square delimitated on FIG. 7N.

FIG. 7P is a top view μCT scan of PDN₁.

FIG. 8A is a SEM image of pure gelatin.

FIG. 8B is a SEM image of gelatin-PDN.

FIG. 8C is a bar graph of the pore size for pure gelatin and gelatin-PDN hydrogels (*** represents P<0.001).

FIG. 8D is a photograph of pure gelatin specimens ruptured during sample preparation steps due to the brittleness of the hydrogel matrix.

FIG. 8E is a photograph of gelatin-PDN that demonstrates its stretchability (λ=1).

FIG. 8F is a photograph of gelatin-PDN that demonstrates its stretchability (λ˜2.35).

FIG. 8G is a bar graph of the toughness of pure gelatin compared to gelatin-PDN.

FIG. 9A is a schematic of the permeability measurement, the height and cross-sectional area of the gel are denoted as L and A, respectively, the flow velocity, up- and down-steam pressures are denoted as v, P₀, and P₁.

FIG. 9B is a graph showing the pressure gradient-velocity relations of PDN_{0.5}, PDN₁ and PDN₂ (2 w/v percentage of glycol-chitosan).

FIG. 9C is a bar graph of the permeability for NSN, PSN, PDN_{0.5}, PDN₁ and PDN₂ (**** represents P<0.0001).

FIG. 9D is a bar graph of the permeability for skin, bone, liver, vocal fold, PEGDA, gelatin, agarose, and PDN.

FIG. 9E is a photograph showing the stretchability of NSN (λ=1).

FIG. 9F is a photograph showing the stretchability of NSN (λ˜1.45).

FIG. 9G is a photograph showing the stretchability of PDN₁ (λ=1).

FIG. 9H is a photograph showing the stretchability of PDN₁ (λ˜2.64).

FIG. 9I is a graph showing the stress in function of stretch for NSN, PSN and PDN₁.

FIG. 9J is a bar graph showing the toughness of NSN, PSN, PDN_{0.5}, PDN₁ and PDN₂ (** represents P<0.01, **** represents P<0.0001 and n.s. is P>0.05).

FIG. 9K is bar graph showing the stretchability of NSN, PSN, PDN_{0.5}, PDN₁ and PDN₂ (** represents P<0.01 and **** represents P<0.0001).

FIG. 9L is a bar graph showing the fractocohesive length of NSN, PSN, PDN_{0.5}, PDN₁ and PDN₂ (*** represents P<0.001 and **** represents P<0.0001).

FIG. 10A is a schematic of the internal structure of the perfusion chamber.

FIG. 10B is a photograph of the perfusion chamber assembled.

FIG. 10C is a photograph of the perfusion chamber disassembled

FIG. 11 is a bar graph showing the toughness of NSN, PSN, PDN_{0.5}, PDN₁ and PDN₂.

FIG. 12 is a graph of the swelling ratio in function of time for NSN (♦), PSN (•), PDN_{0.5} (▪), PDN₁ (▴) and PDN₂ (▾).

FIG. 13 is a graph of the biodegradation (remaining weight) of NSN (♦), PSN (•), PDN_{0.5} (▪), PDN₁ (▴) and PDN₂ (▾) over time when exposed to an enzyme solution.

FIG. 14A is a confocal image of live/dead stained cells cultured within a NSN hydrogel on Day 0.

FIG. 14B is a confocal image of live/dead stained cells cultured within a PDN₁ hydrogel on Day 0.

FIG. 14C is a confocal image of live/dead stained cells cultured within a NSN hydrogel on Day 7.

FIG. 14D is a confocal image of live/dead stained cells cultured within a PDN₁ hydrogel on Day 7.

FIG. 14E shows a bar graph of the viability of cells on hydrogels at culture days 0, 3, and 7 (NSN ( [symbol] ), PSN ( [symbol] ), PDN_{0.5} ( [symbol] ), PDN₁ ( [symbol] ) and PDN₂( [symbol] )).

FIG. 14F shows a bar graph of the normalized cell density at culture days 0 and 7 in hydrogels (NSN ( [symbol] ), PDN ( [symbol] ), PDN_{0.5} ( [symbol] ), PDN₁ ( [symbol] ) and PDN₂ ( [symbol] )) (n.s represents P >0.05, *<represents P<0.1, ** represents P<0.01, and *** represents P<0.001).

FIG. 14G is a confocal image of the morphology of cells culture in NSN hydrogel at culture day 7.

FIG. 14H is a confocal image of the morphology of cells culture in PDN₁ hydrogel at culture day 7.

FIG. 14I shows a bar graph of the circularity of NSN, PSN, PDN_{0.5}, PDN₁ and PDN₂ on day 7 of culture.

FIG. 15A is a live/dead staining confocal image of hVFF cells encapsulated inside NSN at day 0.

FIG. 15B is a live/dead staining confocal image of hVFF cells encapsulated inside PSN at day 0.

FIG. 15C is a live/dead staining confocal image of hVFF cells encapsulated inside PDN_{0.5} at day 0.

FIG. 15D is a live/dead staining confocal image of hVFF cells encapsulated inside PDN₁ at day 0.

FIG. 15E is a live/dead staining confocal image of hVFF cells encapsulated inside PDN₂ at day 0.

FIG. 15F is a live/dead staining confocal image of hVFF cells encapsulated inside NSN at day 3.

FIG. 15G is a live/dead staining confocal image of hVFF cells encapsulated inside PSN at day 3.

FIG. 15H is a live/dead staining confocal image of hVFF cells encapsulated inside PDN_{0.5} at day 3.

FIG. 15I is a live/dead staining confocal image of hVFF cells encapsulated inside PDN₁ at day 3.

FIG. 15J is a live/dead staining confocal image of hVFF cells encapsulated inside PDN₂ at day 3.

FIG. 15K is a live/dead staining confocal image of hVFF cells encapsulated inside NSN at day 7.

FIG. 15L is a live/dead staining confocal image of hVFF cells encapsulated inside PSN at day 7.

FIG. 15M is a live/dead staining confocal image of hVFF cells encapsulated inside PDN_{0.5} at day 7.

FIG. 15N is a live/dead staining confocal image of hVFF cells encapsulated inside PDN₁ at day 7.

FIG. 15O is a live/dead staining confocal image of hVFF cells encapsulated inside PDN₂ at day 7.

FIG. 16A is a confocal image of the morphology (F-actin and nuclei stained) of NSN at day 7 of culture.

FIG. 16B is a confocal image of the morphology (F-actin and nuclei stained) of PSN at day 7 of culture.

FIG. 16C is a confocal image of the morphology (F-actin and nuclei stained) of PDN_{0.5} at day 7 of culture.

FIG. 16D is a confocal image of the morphology (F-actin and nuclei stained) of PDN₁ at day 7 of culture.

FIG. 16E is a confocal image of the morphology (F-actin and nuclei stained) of PDN₂ at day 7 of culture.

FIG. 17A is a schematic of the experimental set up for the cell penetration assay.

FIG. 17B is a confocal image showing hVFFs penetration into a NSN hydrogel. Cells were counterstained with DAPI.

FIG. 17C is a confocal image showing hVFFs penetration into a PDN hydrogel. Cells were counterstained with DAPI.

FIG. 17D is a bar graph of the cell penetration depth for NSN and PDN.

FIG. 18A is a schematic showing the delivery of PDN to microfluidic channels through injection.

FIG. 18B is a photograph of a hydrogel injection.

FIG. 18C is a photograph of a media perfusion though the hydrogels of FIG. 18B.

FIG. 18D is a confocal image showing live/dead cells within PDN without perfusion after 24 hours.

FIG. 18E is a confocal image showing live/dead cells within PDN after 24 hours of perfusion at the inlet.

FIG. 18F is a confocal image showing live/dead cells within PDN after 24 hours of perfusion at the middle.

FIG. 18G is a confocal image showing live/dead cells within PDN after 24 hours of perfusion at the outlet.

FIG. 18H is a bar graph of the cell viability in different microfluidic channel conditions (no perfusion, at the inlet with perfusion, in the middle with perfusion, and at the outlet with perfusion).

FIG. 18I is a photograph of a PDN-enabled microfluidic setup within a standard 6-well plate.

FIG. 18J is a photograph showing the microfluidic channels before perfusion.

FIG. 18K is a photograph showing the different microfluidic channels during perfusion.

FIG. 18L is a photograph showing injected PDNs taken out of the microfluidic channel and handled without rupture.

FIG. 19A is a photograph showing the design and setup of a phonomimetic bioreactor.

FIG. 19B is a schematic top view showing the design and setup of a phonomimetic bioreactor.

FIG. 19C is a schematic cross section view showing the design and setup of a phonomimetic bioreactor.

FIG. 19D is a photograph of PDN hydrogel before injection into the phonomimetic bioreactor.

FIG. 19E is a photograph showing the PDN hydrogel being injected into the phonomimetic bioreactor.

FIG. 19F is a photograph showing the perfusion of the PDN hydrogel of FIG. 19E.

FIG. 19G is a graph of the subglottal pressure profiles applied on the injected hydrogels in the vocal fold in function of time.

FIG. 19H is a graph showing the daily high-frequency cyclic mechanical stimulations that a hydrogel would be subject to in the vocal fold.

FIG. 19I is a photograph of the movement of bioreactor lips within one cycle from the top-down view (open configuration).

FIG. 19J is a photograph of the movement of bioreactor lips within one cycle from the top-down view (half-open configuration).

FIG. 19K is a photograph of the movement of bioreactor lips within one cycle from the top-down view (closed configuration).

FIG. 19L is a photograph showing the morphologies of injected hydrogels (NSN, PSN, and PDN) after mechanical stimulation.

FIG. 19M shows COMSOL™ images of simulated morphologies of injected hydrogels after mechanical stimulations.

FIG. 19N is a graph showing the stiffness of pristine PDN and PDN after being stimulated in the bioreactor for 7 days (n.s. is P>0.05).

FIG. 19O is a confocal image showing the live/dead cells cultured within injected PDN at day 0.

FIG. 19P is a confocal image showing the live/dead cells cultured within injected PDN after photonation for 7 days.

FIG. 20A is a SEM image of porous PDN before phonation inside the bioreactor for over 1 million cycles under perfusion.

FIG. 20B is a SEM image of porous PDN after phonation inside the bioreactor for over 1 million cycles under perfusion.

FIG. 21 shows a finite element analysis (FEA) simulation showing the stress distribution of PDN and the elastomeric parts of the bioreactor during one period of oscillation. t/T represents the normalized time during one period. Black contours indicate the undeformed shape and the inner circle refers to the hydrogel.

FIG. 22A is a confocal image of collagen secretion by hVFF cells within NSN (static).

FIG. 22B is a confocal image of collagen secretion by hVFF cells within PDN (static).

FIG. 22C is a confocal image of collagen secretion by hVFF cells within PDN (phonated).

FIG. 23A is a photograph before the injection of PDN into a rat cadaver.

FIG. 23B is a photograph of the injection of PDN (0.85 mL) into a rat cadaver subcutaneously through a fine needle (21G).

FIG. 23C is a schematic of the use of PDN for vocal fold repair and regeneration showing the vocal fold lesions removal.

FIG. 23D is a schematic of the use of PDN for vocal fold repair and regeneration showing the microflap closure.

FIG. 23E is a schematic of the use of PDN for vocal fold repair and regeneration showing the PDN injection for wound repair.

FIG. 24A is a confocal microscopy image of NSN (green represents the chitosan network and red represents the gelatin network).

FIG. 24B is a confocal microscopy image of PSN (green represents the chitosan network and red represents the gelatin network.

FIG. 24C is a confocal microscopy image of PDN prepared with 1.25% gelatin and 2.5% chitosan in 0.2 M acetic acid and low enzyme concentration (green represents the chitosan network and red represents the gelatin network).

FIG. 24D is a confocal microscopy image of PDN prepared with 1.25% gelatin and 2.5% chitosan in 0.2 M acetic acid and high enzyme concentration (green represents the chitosan network and red represents the gelatin network).

FIG. 24E is a confocal microscopy image of PDN prepared with 2.50% gelatin and 2.5% chitosan in 0.2 M acetic acid and low enzyme concentration (green represents the chitosan network and red represents the gelatin network).

FIG. 24F is a confocal microscopy image of PDN prepared with 2.50% gelatin and 2.5% chitosan in 0.2 M acetic acid and high enzyme concentration (green represents the chitosan network and red represents the gelatin network).

FIG. 24G is a confocal microscopy image of PDN prepared with 3.75% gelatin and 2.5% chitosan in 0.2 M acetic acid and low enzyme concentration (green represents the chitosan network and red represents the gelatin network).

FIG. 24H is a confocal microscopy image of PDN prepared with 3.75% gelatin and 2.5% chitosan in 0.2 M acetic acid and high enzyme concentration (green represents the chitosan network and red represents the gelatin network).

FIG. 24I is a confocal microscopy image of PDN prepared with 5.0% gelatin and 2.5% chitosan in 0.2 M acetic acid and low enzyme concentration (green represents the chitosan network and red represents the gelatin network).

FIG. 24J is a confocal microscopy image of PDN prepared with 5.0% gelatin and 2.5% chitosan in 0.2 M acetic acid and high enzyme concentration (green represents the chitosan network and red represents the gelatin network).

FIG. 25A is a SEM image of NSN (green represents the chitosan network and red represents the gelatin network).

FIG. 25B is a SEM image of PSN (green represents the chitosan network and red represents the gelatin network.

FIG. 25C is a SEM image of PDN prepared with 1.25% gelatin and 2.5% chitosan in 0.2 M acetic acid and low enzyme concentration (green represents the chitosan network and red represents the gelatin network).

FIG. 25D is a SEM image of PDN prepared with 1.25% gelatin and 2.5% chitosan in 0.2 M acetic acid and high enzyme concentration (green represents the chitosan network and red represents the gelatin network).

FIG. 25E is a SEM image of PDN prepared with 2.50% gelatin and 2.5% chitosan in 0.2 M acetic acid and low enzyme concentration (green represents the chitosan network and red represents the gelatin network).

FIG. 25F is a SEM image of PDN prepared with 2.50% gelatin and 2.5% chitosan in 0.2 M acetic acid and high enzyme concentration (green represents the chitosan network and red represents the gelatin network).

FIG. 25G is a SEM image of PDN prepared with 3.75% gelatin and 2.5% chitosan in 0.2 M acetic acid and low enzyme concentration (green represents the chitosan network and red represents the gelatin network).

FIG. 25H is a SEM image of PDN prepared with 3.75% gelatin and 2.5% chitosan in 0.2 M acetic acid and high enzyme concentration (green represents the chitosan network and red represents the gelatin network).

FIG. 25I is a SEM image of PDN prepared with 5.0% gelatin and 2.5% chitosan in 0.2 M acetic acid and low enzyme concentration (green represents the chitosan network and red represents the gelatin network).

FIG. 25J is a SEM image of PDN prepared with 5.0% gelatin and 2.5% chitosan in 0.2 M acetic acid and high enzyme concentration (green represents the chitosan network and red represents the gelatin network).

FIG. 26A is a graph of the pore size of NSN, PSN and enzymatically crosslinked PDNs.

FIG. 26B is a graph of the porosity of NSN, PSN and enzymatically crosslinked PDNs.

FIG. 27A is a confocal microscopy image of NSN showing cell morphology and cluster of differentiation (CD206) expression by macrophages after 6 days of encapsulation (red=F-actin, blue=Hoechst, green=CD206).

FIG. 27B is a confocal microscopy image of PSN showing cell morphology and CD206 expression by macrophages after 6 days of encapsulation (red=F-actin, blue=Hoechst, green=CD206).

FIG. 27C is a confocal microscopy image of PDN prepared with 1.25% gelatin and 2.5% chitosan in 0.2 M acetic acid and low enzyme concentration showing cell morphology and CD206 expression by macrophages after 6 days of encapsulation (red=F-actin, blue=Hoechst, green=CD206).

FIG. 27D is a confocal microscopy image of PDN prepared with 1.25% gelatin and 2.5% chitosan in 0.2 M acetic acid and high enzyme concentration showing cell morphology and CD206 expression by macrophages after 6 days of encapsulation (red=F-actin, blue=Hoechst, green=CD206).

FIG. 27E is a confocal microscopy image of PDN prepared with 2.50% gelatin and 2.5% chitosan in 0.2 M acetic acid and low enzyme concentration showing cell morphology and CD206 expression by macrophages after 6 days of encapsulation (red=F-actin, blue=Hoechst, green=CD206).

FIG. 27F is a confocal microscopy image of PDN prepared with 2.50% gelatin and 2.5% chitosan in 0.2 M acetic acid and high enzyme concentration showing cell morphology and CD206 expression by macrophages after 6 days of encapsulation (red=F-actin, blue=Hoechst, green=CD206).

FIG. 27G is a confocal microscopy image of PDN prepared with 3.75% gelatin and 2.5% chitosan in 0.2 M acetic acid and low enzyme concentration showing cell morphology and CD206 expression by macrophages after 6 days of encapsulation (red=F-actin, blue=Hoechst, green=CD206).

FIG. 27H is a confocal microscopy image of PDN prepared with 3.75% gelatin and 2.5% chitosan in 0.2 M acetic acid and high enzyme concentration showing cell morphology and CD206 expression by macrophages after 6 days of encapsulation (red=F-actin, blue=Hoechst, green=CD206).

FIG. 27I is a confocal microscopy image of PDN prepared with 5.0% gelatin and 2.5% chitosan in 0.2 M acetic acid and low enzyme concentration showing cell morphology and CD206 expression by macrophages after 6 days of encapsulation (red=F-actin, blue=Hoechst, green=CD206).

FIG. 27J is a confocal microscopy image of PDN prepared with 5.0% gelatin and 2.5% chitosan in 0.2 M acetic acid and high enzyme concentration showing cell morphology and CD206 expression by macrophages after 6 days of encapsulation (red=F-actin, blue=Hoechst, green=CD206).

FIG. 28A is a bar graph showing the circularity of macrophages after 6 days of encapsulation in NSN, PSN or enzymatically crosslinked PDNs (**** represents P<0.0001, Sample size, N=3).

FIG. 28B is a bar graph showing the quantification of CD206 expression of macrophages after 6 days of encapsulation in NSN, PSN or enzymatically crosslinked PDNs (**** represents P<0.0001, Sample size, N=3).

FIG. 29A is a photograph of the peeling fronts of PDN from porcine heart tissue.

FIG. 29B is a photograph of the peeling fronts of PDN from porcine skin tissue.

FIG. 29C is a bar graph showing the adhesion energy of PDN to a collagen casing, porcine heat and porcine skin (sample size N=5).

FIG. 30A is a photograph showing the hemostasis ability of PDN when applied to a bleeding rat tail.

FIG. 30B is a bar graph showing the time to hemostasis when no treatment is applied, Gauze standard treatment is applied or PDN treatment is applied (** represents P<0.01, ***P<0.001, n.s. means P≥0.05).

FIG. 30C is is a bar graph showing the blood loss when no treatment is applied, Gauze standard treatment is applied or PDN treatment is applied (** represents P<0.01, n.s. means P≥0.05)

### DETAILED DESCRIPTION

Biological tissues hinge on blood perfusion and mechanical toughness to function. Injectable hydrogels that possess both high permeability and toughness have profound impacts on regenerative medicine but remain a long-standing challenge. To address this issue, the present disclosure provides a porous double-network hydrogel (PDN) that is injectable. In some embodiments, the PDN of the present disclosure gels in situ i.e. after being injected at the desired in vivo site. The PDN can be fabricated by orchestrating stepwise gelation and phase separation processes. The interconnected pores of the resulting hydrogels enable direct medium perfusion through organ-sized matrices. The hydrogels are amenable to cell encapsulation and delivery while promoting cell proliferation and spreading. They are also pore-insensitive, tough, and fatigue-resistant. When tested in biomimetic perfusion bioreactors, the hydrogels maintain physical integrity under prolonged, high-frequency biomechanical stimulations (>6,000,000 cycles at 120 Hz). The PDN of the present disclosure exhibits chemical and physical characteristics that allow it to be injected in stress intense sites such as the vocal folds. These properties also allow for manipulations in tissue engineering, biofabrication, organs-on-chips, drug delivery, and disease modeling.

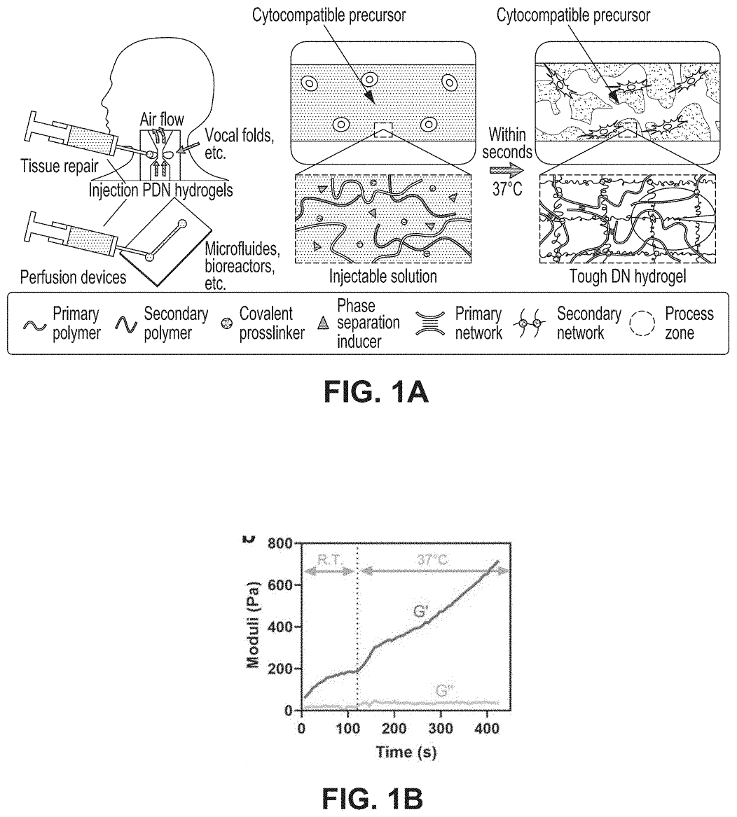

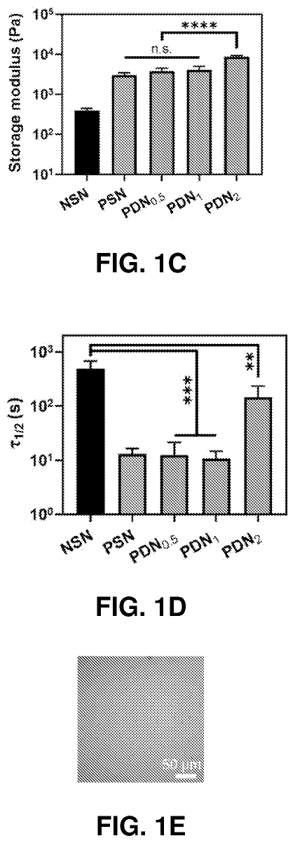

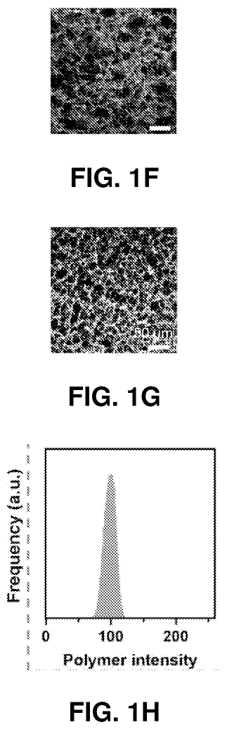

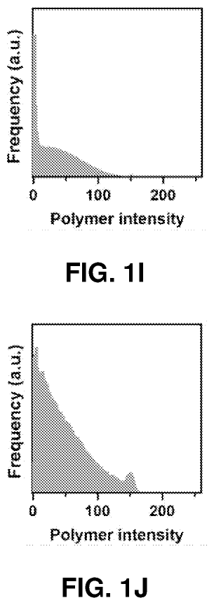

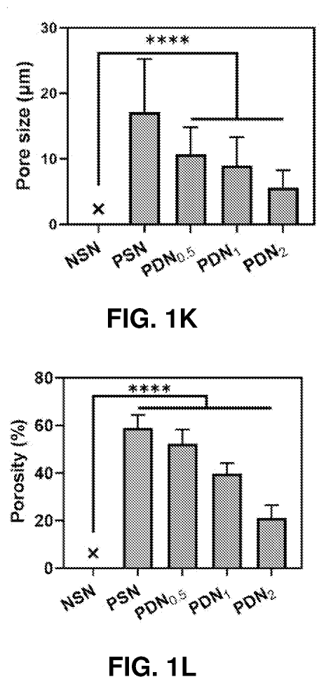

The PDN of the present disclosure comprises a first network comprising a first polymer that can be physically self crosslinked. The term “physically” as used herein in the context of crosslinking refers to physical interactions including but not limited to inter molecular electrostatic bonds such as hydrogen bonds. The PDN of the present disclosure has a second network comprising a second polymer that can be covalently crosslinked. The PDN of the present disclosure differs from previously reported injectable hydrogels that consist of either nanoporous or preformed porous networks. PDNs can form interconnected cell-sized pores in-situ upon injection (FIG. 1A). Unlike many porous hydrogels that are weakened by their pores, PDNs are tough and resilient to millions of cycles of mechanical loadings despite the presence of defect-like pores. They exhibit improved fracture toughness and stretchability compared to nanoporous or porous single-network counterparts, which are termed NSN and PSN, respectively. Both the composition and the gelation method of PDNs are cytocompatible. The highly porous matrices enable rapid media perfusion and support cell spreading and trafficking. Such perfusable hydrogels can support cell survival in organ-sized scaffolds with a dimension beyond 60 mm. Thanks to the facile injectability, they can be easily delivered through fine needles and incorporated into 3D cell culture perfusion systems with complex mechanical loadings, such as microfluidic chips and bioreactors. The PDNs can withstand over 6,000,000 cycles of high-frequency biomechanical stimulations without rupture. The advantageous combination of interconnected pores, toughness, cytocompatibility, and injectability, the described material systems and method render the PDN a desirable medium for regenerative medicine, and biomimetic in vitro 3D cell culture platforms.

The first polymer can be a polymer that exhibits phase separation in a physiological pH range and at body temperature. In such cases the injection of biological material can be performed prior to gelation to then have an in situ gelation. Indeed, phase separation promotes cytocompatibility and in situ pore-forming mechanisms. In some embodiments, the first polymer has a pKₐ of from 6 to 6.5, or from 6.3 to 6.5. The first polymer can be a thermogelling polymer such as chitosan (pKₐ about 6.5) or poly(N-isopropylacrylamide) (pKₐ about 6). When the pH of an acidic first polymer solution is raised above its pKₐ, bicontinuous polymer-rich and polymer-poor phases emerge (i.e. the phase separation). When the polymer-rich phase crosslinks, the polymer-poor phase, comprised mainly of water, results in interconnected open space. This pore-forming mechanism can occur at physiological conditions, without additional chemical reagents, and is suitable for cell encapsulation and delivery. Functional groups such as NH₂ and OH groups contribute to the physical crosslinking by providing hydrogen bonds. This self-crosslinking behavior can stabilize the polymer-rich phase and thereby reinforce the already-formed porous structure. In some embodiments, the first network contains a large number of hydrogen bonds and other intermolecular interactions that can be exploited for energy dissipation as the dissipative primary network. In some embodiments, at least 50% of the primary amine groups on the first polymer (e.g. chitosan) are deprotonated and can form hydrogen bonds. In further embodiments, the first polymer can be alginate, cellulose or poly(4-aminostyrene). In these further embodiments embodiments, if cells are to be incorporated in the PDN, the gelation occurs ex vivo prior to reaching the biological tissue. This is because the gelation pH compromises the cell survivability upon a prolonged exposure.

The second polymer is a polymer capable of covalent crosslinking that is also biocompatible. Polymers that do not affect the phase separation of the first polymer are suitable as second polymers, such as polymers that do not form intrinsic chemical reactions with the first polymer. In some embodiments, the second polymer is a neutral charge polymer. These intrinsic reactions would limit the mobility of the first polymer chains and thus their phase separation. In some embodiments, the second polymer is polyacrylamide, collagen, fibrin, polyethylene glycol, glycol-chitosan or gelatin. In some embodiments, the weight to volume ratio of the weight of the first polymer to the volume of the hydrogel is from 0.25 to 4%, from 0.25 to 3.5%, from 0.25 to 3 or from 0.25 to 2.5%. In some embodiments, the weight to volume ratio of the weight of the second polymer to the volume of the hydrogel is from 0.15 to 8%, from 0.15 to 4%, from 0.15 to 3%, from 0.15 to 2%, from 0.15 to 1.5%, from 0.625 to 4%, from 0.625 to 3%, from 0.625 to 2%, from 0.625 to 1.5%, or from 0.625 to 2.5%.

In some embodiments, the PDN has pores having a diameter of at least 0.1, 0.5, 1, 2, 3, 4, 5, or 6 μm. In further embodiments, the PDN has pores having a diameter of from 0.1 to 100 μm, from 0.5 to 100 μm, from 1 to 100 μm, from 3 to 100 μm, from 6 to 100 μm, from 0.1 to 75 μm, from 0.5 to 75 μm, from 1 to 75 μm, from 3 to 75 μm, from 6 to 75 μm, from 0.1 to 50 μm, from 0.5 to 50 μm, from 1 to 50 μm, from 3 to 50 μm, from 6 to 50 μm, from 0.1 to 25 μm, from 0.5 to 25 μm, from 1 to 25 μm, from 3 to 25 μm, from 6 to 25 μm, from 0.1 to 15 μm, from 0.5 to 15 μm, from 1 to 15 μm, from 3 to 15 μm, from 6 to 15 μm, from 0.1 to 10 μm, from 0.5 to 10 μm, from 1 to 10 μm, from 3 to 10 μm, or from 6 to 10 μm. The porosity of the PDN hydrogel can be, in one example, from 18 to 70%, from 21 to 70%, from 18 to 54% or from 21 to 54%. As will be described in further details herein below, the PDN of the present disclosure is tough and can be characterized by a fracture toughness of at least 5, at least 10, or at least 20 J m⁻². In some embodiments, the fracture toughness is from 5 to 39, from 10 to 39, or from 20 to 39 J m⁻². The PDN of the present disclosure exhibits a desirable permeability that promotes cell survival in the hydrogel matrix. For example, the PDN can have a permeability of from 10⁻¹⁴ to 10⁻¹² m². Other parameters such as the half-life time of stress relaxation, stretchability, and fractocohesive length further define the desirable mechanical properties exhibited by PDN. In one embodiment, the half-life time of stress relaxation from 10 to 100 s. In a further embodiment, a stretchability of at least 3. In yet a further embodiment, the fractocohesive length of from 0.25 mm to 0.5 mm, from 0.3 mm to 0.5 mm or 0.4 mm to 0.5 mm.

There is provided a method of fabricating the PDN of the present disclosure. The method comprises providing a precursor solution containing precursors of the first and second polymers. A basic gelling agent solution is also provided, for example a solution containing a bicarbonate salt and glyoxal, or alternatively a phosphate salt and glyoxal. In some embodiments, the gelling agent is selected from sodium phosphate dibasic, sodium phosphate monobasic, sodium bicarbonate, calcium phosphate dibasic, potassium phosphate monobasic, potassium phosphate dibasic, β-Glycerol phosphate disodium and combinations thereof. The precursor solution is mixed with the basic gelling agent solution to raise the pH of the mixture from acidic to neutral. This induces the phase separation of the first polymer to initiate the formation of the first network also referred to as the dissipative network. In some embodiments, the volume ratio of the precursor solution to the basic gelling solution is from 1:1 to 2:1, preferably about 3:2 where “about” is defined as ±15%, preferably ±10%, more preferably ±5%. Following that, chemical crosslinking of the second polymer occurs to form the secondary network. In one example, the chemical crosslinking of the second polymer is a self-crosslinking reaction that forms covalent bonds. In a further example, the chemical crosslinking of the second polymer is an enzymatic crosslinking that forms covalent bonds such as peptide bonds. In embodiments where the second polymer is crosslinked via enzymatic crosslinking, the enzyme is provided with the basic gelling agent solution, for example in a concentration of from 10 to 500 mg/mL, from 25 to 400 mg/mL or from 50 to 250 mg/mL. Examples of suitable enzymes include but are not limited to transglutaminase, horseradish peroxidase and radical S-adenosylmethionine (rSAM). Indeed, horseradish peroxidase and rSAM can crosslink gelatin. Cells can be incorporated into the gelation procedure before injection into a site. In some embodiments, the cells are incorporated in the hydrogel after the pH has reached a value higher than 6.

Without wishing to be bound by theory, the gelation of the PDNs of the present disclosure involves three coordinated processes: initial solidification, phase separation and further crosslinking. The initial solidification is reliant on the thermogelling behavior of the first polymer. Accompanying the initial solidification, phase separation generally takes place within seconds of the initial solidification. Sequentially, the second polymer requires around 15 minutes before starting to crosslink covalently.

The PDN of the present disclosure can be used as a hemostatic agent for the treatment of hemorrhages and injuries. PDN can be combined with other therapeutic agents, for example a therapeutic agent can be encapsulated within the matrix of PDN. The pore size of the PDN of the present disclosure is suited for cell culture and cell encapsulation. Accordingly, the PDN of the present disclosure can be used in cellular therapy, organ regeneration, organ remodeling, drug delivery, cell delivery, cancer vaccine immunotherapy, microfluidic cell culture, mechanobiology research and organ-on-chips.

### EXAMPLE

Hydrogel synthesis: Chemicals used in the present example were purchased from Sigma-Aldrich™ and used without further purification unless stated otherwise. Chitosan (DDA: 95%, medium and high molecular weight) was purchased from Xi'an Lyphar™ Biotech. Pure chitosan (PC) powder was dissolved and stirred in 0.2 M acetic acid to form a homogeneous chitosan solution. Different concentrations of glycol-chitosan purchased from Sigma-Aldrich™ (GC, G7753) were added to the chitosan solution to form PDN precursors. To prepare the gelling agents, a phosphate solution (PS) was firstly prepared by mixing 0.1 M sodium phosphate dibasic (Na₂HPO₄, S7907) and 0.1 M sodium phosphate monobasic (NaH₂PO₄, S8282) with a volume ratio of 50:3. The gelling solutions were then completed by adding glyoxal and sodium bicarbonate (SC, S233-500, Fisher Scientific™) into the phosphate solution. A hydrogel precursor and its associated gelling agent were mixed at a volume ratio of 3:2 using a syringe connector to yield hydrogels. Materials for synthesizing control groups included alginate (ULV-L3G, KIMICA™ Corporation), gelatin type A (G2500), and CaSO₄ (C3771). The detailed ingredients for each formulation are listed in Table 1 below:

TABLE 1
Hydrogel synthesis reagents

| | Hydrogel precursor | Gelling agent |
|---|---|---|
| NSN | 3.33% GC in PBS | 0.0124% glyoxal in PBS |
| PSN | 2.5% PC in 0.2M acetic acid | 0.445M SC in PS |
| PDN0.5 | 0.84% GC + 2.5% PC in 0.2M | 0.445M SC+ 0.0031% |
| | acetic acid | glyoxal in PS |
| PDN1 | 1.67% GC + 2.5% PC in 0.2M | 0.445M SC + 0.0062% |
| | acetic acid | glyoxal in PS |
| PDN2 | 3.34% GC + 2.5% PC in 0.2M | 0.445M SC + 0.0124% |
| | acetic acid | glyoxal in PS |
| Pure | 4.17% gelatin in PBS | 0.0155% glyoxal in PBS |
| gelatin | | |
| Gelatin- | 1.67% gelatin + 2.5% PC in | 0.445M SC + 0.0062% |
| PDN | 0.2M acetic acid | glyoxal in PS |
| NDN | 1.67% GC + 2.5% alginate in | 0.1M CaSO₄+ 0.0062% |
| | DI water | glyoxal in water |

More specifically, to synthesize PDN_{0.5}, glycol chitosan and chitosan were first added and dissolved together into 0.2 M acetic acid solution at a concentration of 0.84% and 2.5%, respectively. The solution was used as the hydrogel precursor. Then a phosphate solution was prepared by mixing 0.1 M sodium phosphate dibasic solution with 0.1 M sodium phosphate monobasic solution at a volume ratio of 50:3. Sodium bicarbonate and glyoxal were added to the prepared phosphate solution at a concentration of 0.445 M and 0.0031%, respectively. The resulting solution was used as the gelling agent. PDN_{0.5} is formed by mixing the hydrogel precursor and the gelling agent at a volume ratio of 3:2 using a syringe connector.

To synthesize PDN₁, glycol chitosan and chitosan were first added and dissolved together into 0.2 M acetic acid solution at a concentration of 1.67% and 2.5%, respectively. The solution was used as the hydrogel precursor. Then a phosphate solution was prepared by mixing 0.1 M sodium phosphate dibasic solution with 0.1 M sodium phosphate monobasic solution at a volume ratio of 50:3. Sodium bicarbonate and glyoxal were added to the prepared phosphate solution at a concentration of 0.445 M and 0.0062%, respectively. The resulting solution was used as the gelling agent. PDN₁ is formed by mixing the hydrogel precursor and the gelling agent at a volume ratio of 3:2 using a syringe connector.

To synthesize PDN₂, glycol chitosan and chitosan were first added and dissolved together into 0.2 M acetic acid solution at a concentration of 3.34% and 2.5%, respectively. The solution was used as the hydrogel precursor. Then a phosphate solution was prepared by mixing 0.1 M sodium phosphate dibasic solution with 0.1 M sodium phosphate monobasic solution at a volume ratio of 50:3. Sodium bicarbonate and glyoxal were added to the prepared phosphate solution at a concentration of 0.445 M and 0.0124%, respectively. The resulting solution was used as the gelling agent. PDN₂ can be formed by mixing the hydrogel precursor and the gelling agent at a volume ratio of 3:2 using a syringe connector.

Mechanical characterizations: Gelation kinetics and frequency sweeps were measured using a torsional rheometer (HDR-2™, TA Instruments) with parallel plates (upper plate diameter of 20 mm). The shear moduli of hydrogels were obtained from isothermal time sweeps at a frequency of 0.1 Hz and 0.1% strain at 37° C. for 2 hours. Frequency sweeps ranging from 0.01-100 Hz at 0.1% strain and 37° C. followed to determine the damping ratios. Relaxation moduli were obtained by holding a step compressive strain of 15% using an Instron™ machine (Model 5965, 10 N load cell) and measuring the compressive stress-time profiles.

The fracture energy or toughness of hydrogels was determined using pure shear tests. One pair of samples was used for each data point. One sample was unnotched, and the other sample was notched. In their undeformed state, each sample had a width W=40 mm and a thickness T=1.5 mm. The distance between the two PET clamps was H=5 mm. The unnotched sample was pulled by an Instron machine with a 10 N load cell at a strain rate of 2 min⁻¹ to measure the stress-stretch (S-λ) curve. For the notched sample, a notch length of ˜10 mm was introduced using a razor blade. The notched sample was pulled until rupture to obtain the critical stretch (λ_{c}). The fracture energy was calculated using S-A curve from the unnotched sample: Γ=H∫₁^{λ}^{_{c}} sdλ.

Structural characterizations: The polymer network was imaged using a confocal microscope (LSM 710™, Zeiss). Both chitosan and glycol-chitosan were conjugated with FITC fluorescent labels. Samples were prepared by mixing fluorescent-labeled polymer solutions and cross-linkers in a vial and transferring ˜150 μL into a 35-mm Petri dish with a coverslip bottom (P35G-0-10-C, MatTek™). Hydrogels were immersed under PBS and imaged as prepared. The polymer network was imaged with 10× and 20× objective lenses.

Macro- and microscopic pores were also imaged using a field emission scanning electron microscope (SEM) (F50, FEI) under various magnifications. Before SEM imaging, all samples were immersed inside 30, 50, 70, 80, 90, and 100% ethanol in sequence for dehydration. Ethanol inside the hydrogels was removed using a CO₂ supercritical point dryer (CPD030™, Leica) to preserve the original pore size. The dehydrated samples were coated 4 nm Pt using a high-resolution sputter coater (ACE600™, Leica) to increase surface conductivity.

Imaging with micro-computed tomography (μCT) was performed using a SkyScanner™ 1172 (Bruker) through a 360° flat-field corrected scan at 30 kV and 112 μA, with a rotational step size of 0.45°, a cross-sectional pixel size of 6.5 μm, and no filter. The samples were prepared and incubated at 37° C. for 24 hours. The volumetric reconstruction (NRecon™, Micro Photonics) was performed with a beam hardening correction of 40%, a ring artifact correction of 4, and an auto-misalignment correction. The 2D and 3D analyses were carried out using Dragonfly™ software and a grayscale intensity range of 50 to 70 (8-bit images) to remove background noise.

Permeability measurement: A customized t-shaped adaptor was 3D printed to enable a controlled application of pressure to force the test fluid through hydrogels. Before testing, a hydrogel was first cured inside the hydrogel container at 37° C. The container was then enclosed by slotting it into the main body and screwing on the retaining cap. The pressure sensor was then connected, and the modified syringe connectors were opened. A liquid-loaded syringe was then connected to the perpendicular port and the adaptor was slowly filled while ensuring all the air escapes. The air outlet was then sealed before the test began. During the test, the syringe pump was set to advance at a fixed rate and the pressure was measured. The fluid that passes through the hydrogel was collected and measured using the stopwatch and bucket method. The measured pressure and volume were used to calculate the permeability of the gel according to Darcy's law Q=−k/μ∇P.

Cell culture: Immortalized hVFFs were cultured in Dulbecco's Modified Eagle Medium (DMEM, Corning) containing sodium pyruvate and supplemented with 10% fetal bovine serum, 1% penicillin/streptomycin, and 1% MEM non-essential amino acids. Cells were incubated at 37° C., in a 5% CO₂ humidified atmosphere. The media were changed every three days for 2D cultures. Cells were disassociated using 0.25% trypsin-EDTA when the cell confluency reached 70%.

Cytocompatibility: To evaluate the cytocompatibility of hydrogels, hVFFs were suspended in hydrogel mixtures immediately after the precursors and gelling agents were mixed. The final cellular concentration was 1 million/mL. The mixtures were then injected into Petri dishes to form hydrogels. Complete DMEM with 10% FBS was used as cell culture medium and changed every day. HVFFs were stained by a LIVE/DEAD viability kit (L3224, Invitrogen™) inside 3D matrices on Day 0, 3, 7. Imaging of fixed hVFFs was conducted using a confocal laser scanning microscope (LSM710™, Zeiss, Germany). Live cells were shown in green fluorescence and dead cells were shown in red.

Cell penetration: To evaluate the cell penetration into the hydrogels, hVFFs cultured in 2D flasks were firstly starved in serum-free DMEM for 6 hours. Cells were then detached and suspended in serum-free DMEM at a concentration of 50,000 cells/mL. 200 μL of cell-free hydrogels were coated to cell culture inserts (08-771-10, Fisher Scientific™) to evenly cover the permeable membrane of 0.45 μm pore size. Serum-free cell suspension (0.8 mL) was added on top of each hydrogel. The cell inserts were then placed into a 12-well plate. Serum-rich DMEM containing 10% FBS was then added to the wells and outside of cell culture inserts to form a chemoattractant gradient across the permeable membrane. Cells were cultured for 2 days before being counterstained with DAPI (D1306, Invitrogen™) using a 1:5000 dilution for 5 min, followed by rinsing twice with PBS. Z-stack imaging of cell penetration into the hydrogels was conducted using a confocal laser scanning microscope (LSM800™, Zeiss, Germany).

Immunohistochemistry: Hydrogels were first washed with pre-warmed PBS twice and then fixed in 3.7% formaldehyde solution for 15 mins. The fixed samples were washed with PBS again twice and permeabilized with 0.1% Triton X-100 in PBS for 5 minutes. The samples were blocked in 1% bovine serum albumin (BSA, A1595) for 1 hour. To conduct F-actin staining, 10 μL of Alexa™ Fluor 633 Phalloidin (A22284, Invitrogen™) was diluted into 200 μL PBS containing 1% BSA. The samples were incubated inside the staining solution at room temperature for 30 mins followed by three times PBS wash. To conduct collagen staining, rabbit polyclonal antibody of collagen-I (1:200, ab34710, Abcam™) was added to PBS containing 1% BSA. The samples were incubated inside the staining solution at room temperature for 30 mins followed by three times PBS wash. The samples were blocked again in goat serum and then incubated for 1 hour with the Alexa™ Fluor 488 goat anti-rabbit IgG secondary antibody (1:1000, A11034, Invitrogen™) followed by three times PBS wash. The nuclei were counterstained with DAPI using a 1:5000 dilution for 5 min, followed by rinsing twice with PBS.

Swelling and biodegradation: The swelling ratios were determined by immersing the hydrogel disks (10 mm in diameter, 1.5 mm in thickness) in PBS (pH=7.4) at 37° C. with gentle mechanical stimulation (75 RPM). The diameters of the disks were measured using a caliper at pre-determined time intervals using a pipette. The swelling ratio was calculated by dividing the measured diameter size by the initial value. For biodegradation assays, all hydrogel samples were prepared with the same volume (500 μL). The average dry weight of the pristine hydrogels was used as the weight at Day 0. After that, an enzyme solution consisting of 13 μg/ml lysozyme (MP Biomedicals™, 100831) in PBS was added to the gels. The samples were incubated at 37° C. with gentle mechanical stimulation over 28 days. The enzyme solution was changed every other day. At pre-determined time intervals, the enzyme solution was removed. The samples were then washed three times for 5 minutes with PBS. The samples were then lyophilized and the remaining polymer dry weight was measured. The remaining ratio of the polymer was calculated by dividing the dry weight of the remaining polymer by the dry weight of the initial gels.

Microfluidic devices: The body of the microfluidic devices was fabricated using soft lithography. In brief, a negative mold was created by printing a Pluronic™ F-127 ink (37 wt % in DI water, P2443) inside a Petri dish into predefined patterns with a bioprinter (BioAssemblyBot™′ Advanced Solutions). PDMS (SYLGARD™ 184, Dow™) was prepared by mixing the base to cure at a weight ratio of 10:1. PDMS was degassed and poured into the Petri dish to cover the printed constructs. After curing at 60° C. overnight, the cured PDMS was taken out of the mold. Pluronic F-127 was removed by washing in cold water. The surfaces of the PDMS body and glass slide were treated with oxygen plasma before bonding to form the complete device. A 2-mm biopsy punch was used to create openings for the inlets and outlets. Devices were repeatedly sterilized with 70% ethanol before washing with PBS. Hydrogels were injected to fill the microfluidic channels. The devices were incubated at 37° C. for 30 mins before flow perfusion.

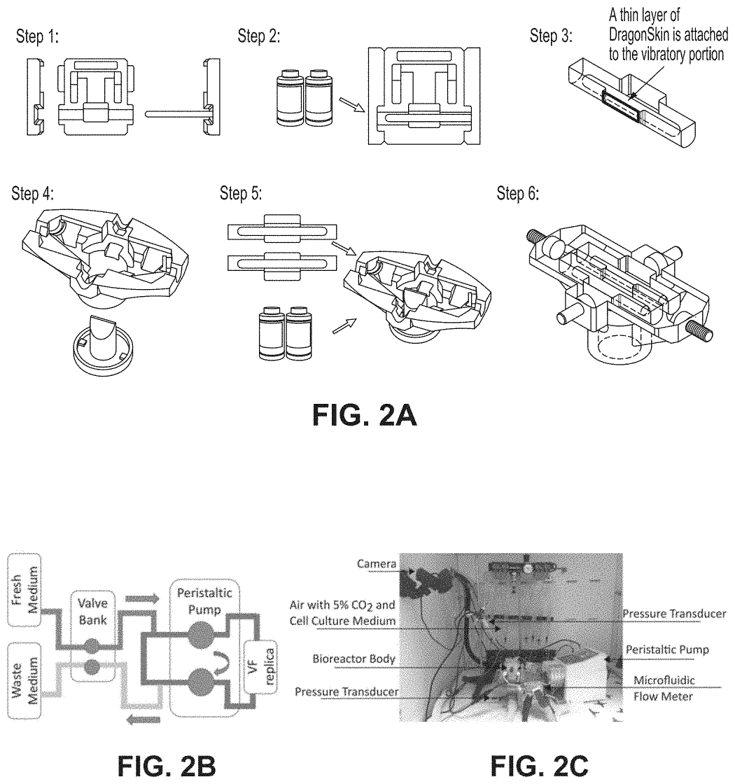

Bioreactor. The bioreactor fabrication steps are illustrated in FIG. 2A. Step 1 shows the preparation of a negative mold with a cylindrical cavity as a vocal fold replica. At step 2, EcoFlex™ 10 was mixed at a weight ration of Part A:Part B:silicone thinner=1:1:1.5. If an automatic mixing machine with a degassing function to remove the bubbles along with the mixing step is used no additional degassing is needed. For manual mixing, a degassing step is needed, for example, centrifuge or vacuum. The resulting degassed mixture was poured into the mold. At step 3, the cured silicone is taken out after 6 hours. The extra parts are trimmed. The cavity is washed with 70% ethanol and sterile water. Step 4 shows the negative mold assembly for fabricating the full bioreactor body. At Step 5, the vocal fold replica is assembled into the negative mold for the bioreactor body. The EcoFlex™ 30 was mixed at a weight ratio Part A:Part B=1:1 and the resulting degassed mixture was poured into the mold. Finally, at step 6, after 6 hours of curing in the mold the bioreactor is ready to use.

Sterile needles (305198, BD Medical™) were first inserted from the two sides of the bioreactor body until reaching the empty hydrogel chamber. Hydrogel precursors and their associated gelling agent were quickly mixed, followed by mixing in a cell suspension to reach a final concentration of 2 million/mL. The cell-laden hydrogel precursors were then injected through pre-inserted needles to fill the chambers. The bioreactor was then placed inside an incubator. Hydrogels were left to crosslink for 2 hours before cell culture media was perfused. The average perfusion flow rate was 5 μL/min. The bioreactor was phonated for 2 hours per day over 7 days. Dynamic subglottal and supraglottal pressure was monitored using two pressure transducers (130D20, PCB Piezotronics™) placed 10 cm below and above the bioreactor lips, respectively. The microphone was connected to a conditioning amplifier (Brüel & Kjaer) that connected to a data acquisition system (National Instruments). Digital readouts for flow and pressure were displayed on a PR 4000F (MKS Instruments). Hydrogels were harvested after pre-determined time points for various assays.

Numerical simulations: COMSOL™ Multiphysics (Stockholm, Sweden) was employed to simulate the phonation in the vocal fold bioreactor. A two-dimensional fully coupled fluid-structure interaction (FSI) model was developed using the unsteady Navier-Stokes equations for the fluid domain. The solid domain consisted of three parts representing the lamina propria (hydrogels), vocalis muscle (Ecoflex™ 00-10), and a thin epithelium layer (Dragon Skin). The hyperelastic Ogden material model was used for the solid domain. The strain energy density for the Ogden model is given by

$${ψ}_{D} = \frac{μ}{α} ( {λ}_{1}^{α} + {λ}_{2}^{α} + {λ}_{3}^{α} - 3 ) \qquad ( 1 )$$

where ψ_{D} is the strain energy density, μ and α are the fitting coefficients, and λᵢ is the ith principal stretch. The nominal stress S for a pure shear test is given by

S=μ(λ^{α-1}−λ^{-(α+1)}) (2)

The Ogden parameters for the hydrogels were determined by fitting Eq. (2) to the loading paths in the pure shear test results of the hydrogels. The Ogden parameters for the Ecoflex™ 00-10 and Dragon Skin were extracted from our previous measurement.

Rayleigh damping model was used for the hydrogels. A dynamic system can be described by

$$[ M ] \frac{{d}^{2} x}{{dt}^{2}} + [ C ] \frac{dx}{dt} + [ K ] x = {F}_{s t a t i c} + {F}_{dynamic} \qquad ( 3 )$$

where [M] is the mass matrix, [C] is the damping matrix, [K] is the stiffness matrix, x is displacement as a function of time, and F_{static} and F_{dynamic}, are static and dynamic loads, respectively. The system damping matrix is defined by

[C]=δ[M]+β[K] (4)

where δ and β are the mass and stiffness proportional Rayleigh damping coefficients, respectively. The Rayleigh damping coefficients were determined by the different damping ratios ξ at different response frequencies co in rad/s according to

$$ξ = \frac{1}{2} ( \frac{δ}{ω} + β ω ) \qquad ( 5 )$$

The damping ratios,

$$ξ = \frac{1}{2} \frac{{G}^{″}}{{G}^{′}} ,$$

were measured from a frequency sweep between 0.1 to 100 Hz using a rheometer, where G′ and G″ are the storage and loss moduli, respectively. Damping in the two other solid bodies was modeled using the isotropic loss factor to account for the intrinsic damping properties of the materials. For the fluid domain, the no-slip boundary condition was applied on the surface of the elastomers. The inlet airflow was defined as an incompressible fully developed laminar flow at room temperature.

A symmetric one half-body of the M5 vocal fold model was designed from a canonical model (R. C. Scherer et al., 2001) for glottal airflow simulation. Dynamic free triangular fine meshes were used to allow for the FSI modeling. An implicit time-dependent fluid solver with a step size of 0.001 s was used in conjunction with a physically controlled tolerance. The stress distribution within the elastomers and the hydrogels were obtained from the simulations. Tables 2 and 3 present the simulation parameters and the material properties, respectively.

TABLE 2
Simulation parameters used in the numerical model

| Parameter | Value |
|---|---|
| Inlet average velocity of air (m s⁻¹) | 2.66 |
| Outlet pressure | 0 |
| Half of the initial glottal gap size (mm) | 1 |
| Epithelium thickness (mm) | 0.1 |
| Minimum mesh size(mm) | 0.0075 |
| Maximum mesh size (mm) | 1.33 |
| Dragon Skin density (kg m⁻³) | 1 070 |
| Dragon Skin dynamic viscosity (Pa · s) | 20 |
| Dragon Skin Young’s modulus (Pa) | 592 949 |
| Dragon Skin Poisson’s ratio | 0.49 |
| Dragon Skin isotropic structural loss factor | 0.24 |
| Ecoflex ™ 00-10 density (kg m⁻³) | 1 040 |
| Ecoflex ™ 00-10 Young’s modulus (Pa) | 9 693 |
| Ecoflex ™ 00-10 Poisson’s ratio | 0.49 |
| Ecoflex ™ 00-10 isotropic structural loss | 0.53 |
| factor | |

TABLE 3
Material parameters of the hydrogels used in simulation.

| Parameter | NSN | PSN | PDN |
|---|---|---|---|
| Density (kg m⁻³) | 1 000 | 1 000 | 1 000 |
| Poisson’s ratio | 0.49 | 0.49 | 0.49 |
| Ogden parameter α | 3.24 | 1.91 | 2.79 |
| Ogden parameter μ (Pa) | 1 913.57 | 2 526.60 | 3 159.89 |
| Mass damping parameter δ | 0.0093 | 0.027 | 0.026 |
| (s⁻¹) | | | |
| Stiffnessdamping | 0.0029 | 0.0021 | 0.0048 |
| parameter β (s) | | | |

Statistical analysis: A sample size of N≥3 was used for all experiments described below. Data are shown as mean± standard deviation (SD). Statistical analysis was performed using one-way ANOVA and post hoc Tukey tests for multiple comparisons or Student's t-tests for comparison between two groups (Prism 9™, GraphPad Inc.). P values <0.05 were considered statistically significant.

The design of PDN proceeded according to the following criteria: i) cytocompatibility, ii) in-situ pore-forming mechanism; and iii) double-network framework. To satisfy the first two criteria, it was hypothesized that the phase separation of cytocompatible biopolymers at body temperature and physiological pH could both ensure cytocompatibility and generate porous structures in-situ. Chitosan was selected, a polysaccharide that exhibits phase separation behavior and finds wide uses in biomedical applications, as an example to test this hypothesis. When the pH of an acidic chitosan solution is raised above its pKₐ, 6.5, bicontinuous polymer-rich and polymer-poor phases emerge. When the polymer-rich phase was crosslinked, the polymer-poor phase, comprised mainly of water, resulted in interconnected open space. This pore-forming mechanism occurs at physiological conditions, without additional chemical reagents, and is suitable for cell encapsulation and delivery. Meanwhile, the primary amine groups [NH₃⁺] of the chitosan deprotonate and are converted to [NH₂], which can bond with the hydroxyl groups [OH] of the chitosan. This self-crosslinking behavior can stabilize the polymer-rich phase and thereby reinforce the already-formed porous structure. Notably, the structure contains a large number of hydrogen bonds and other intermolecular and intramolecular interactions that can be exploited for energy dissipation as the dissipative primary network. To satisfy the third criterion, the secondary network was constructed with biocompatible polymers, such as covalently crosslinked biocompatible polymers. In principle, any polymer that does not affect the phase separation of the dissipative network can be used. In some embodiments, to avoid affecting the phase separation, the first and second polymers are selected such that they do not react with each other to ensure that they do not crosslink in order to maintain the polymer chain mobility. A combination of glycol-chitosan and glyoxal was used. Glycol-chitosan is a derivative of chitosan with improved solubility at neutral pH. It can be crosslinked by dialdehydes such as glyoxal through a Schiff Base reaction to form a secondary network.

To synthesize PDNs, different concentrations of polymer precursor and gelling agent were prepared separately: a PDN precursor comprising chitosan and glycol-chitosan in a weak acetic acid solution; and a gelling agent that contained sodium phosphate monobasic, sodium phosphate dibasic, sodium bicarbonate and glyoxal. Upon mixing of the PDN precursor with the gelling agent, the sodium bicarbonate in the latter raised the pH of the mixture from acidic to neutral and acted as a phase separation inducer to initiate the formation of the dissipative network. Following that, glyoxal chemically crosslinked the glycol-chitosan to form the secondary network. Cells can be incorporated into the mixture before injection. The resulting hydrogels were denoted as PDN, where x stands for the w/v percentage of glycol-chitosan content. The concentration of chitosan for all the conditions was fixed at 1.5% unless indicated otherwise. Nanoporous single-network (NSN) hydrogels containing 2% glyoxal-crosslinked pure glycol-chitosan, and porous single-network (PSN) hydrogels containing 1.5% pure chitosan, were also synthesized as controls. NSN were synthesized according to Ravanbakhsh et al. 2019 and PSN were synthesized according to Bao et al., 2020.

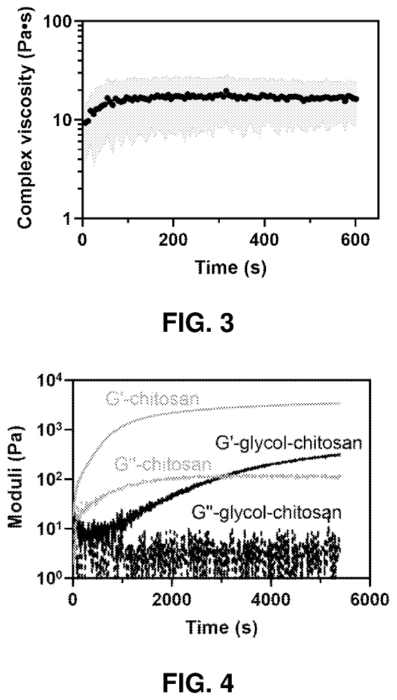

The gelation of injectable, pore-forming hydrogels involved three coordinated processes: initial solidification, phase separation and further crosslinking. The initial solidification should occur in a controlled manner and ensue fast enough to avoid dilution by body fluids. The phase separation and further crosslinking should be separated in time to allow both to proceed independently, leading to interconnected and mechanically stable pores. The PDN hydrogels described herein meet these design criteria. The precursors and gelling agents reacted and partially crosslinked immediately upon mixing, followed by a gradual stiffening process over time. The initial solidification is reliant on the thermogelling behavior of chitosan (i.e., part of secondary network). At room temperature, gelation was slow and steady, allowing time for cell encapsulation and injection (FIG. 1B). The viscosity at room temperature was also low, which is beneficial for injection with reasonable processing time (FIG. 3). After injection and placement at 37° C., gelation accelerated, quickly yielding a strengthened hydrogel (FIG. 1B). Accompanying the initial solidification, phase separation also took place within seconds. Sequentially, the glycol-chitosan needed around 15 minutes before starting to crosslink (FIG. 4). Without wishing to be bound by theory, the disparate kinetics of the fast phase separation and relatively slow covalent crosslinking ensured the mobility of polymer chains before they were immobilized by chemical bonds, which was essential to the formation of a polymer-poor phase for the porous structure.

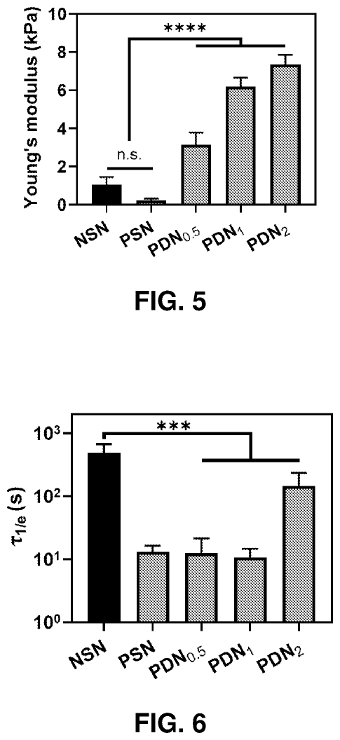

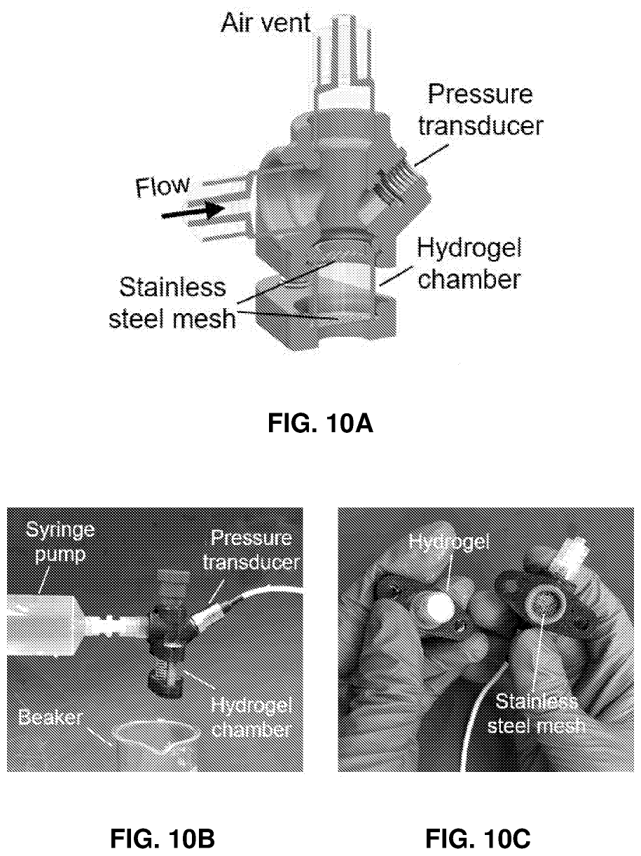

The resulting PDNs showed a favorable viscoelastic response that resembled that of biological tissues. In terms of stiffness, the storage moduli of PDN_{0.5} and PDN₁ were both ˜3.5 kPa and comparable to that of PSN. Additional covalent network polymers further increased the storage moduli to ˜9 kPa (FIG. 10). The stiffness range of PDN spans the range of various biological tissues, such as the vocal folds, lungs, heart, and gastrointestinal tract. For example, the stiffness range can be from 0.1 to 100 kPa or from 0.5 to 15 kPa. Young's moduli of PDNs were also significantly higher than NSN and PSN (FIG. 5). Without wishing to be bound by theory, several possibilities could have contributed to this finding. First, the formation of pores concentrated the polymers and crosslinkers at the solid phase, leading to higher crosslinking density and higher moduli. The potential crosslinking between the chitosan and the glycol-chitosan networks by glyoxal could have further amplified this effect. Second, the polymer concentration of PDN is marginally higher among the three conditions tested, which could have contributed to the concentration effect as well. Third, the Young's moduli were measured at small-strain ranges, whereas the pores mainly affect the large-strain behavior (i.e., because of pore collapse). The synergy of those effects strengthens the PDNs despite their high porosity. Meanwhile, all PDNs exhibited a quick stress relaxation behavior. The stress relaxation behavior was quantified using the half-life time, T_{1/2}, a matrix to relax to one-half of its peak value under a constant compressive strain (15%). Stress relaxation time using the stress retention of 1/e was also evaluated (FIG. 6). Notably, all the PDNs relaxed within 10¹ to 10²s (FIG. 1D). This fast stress relaxation response is comparable to that of organs and native extracellular matrix, such as collagen. This behavior was attributed to the stress-induced rupture of hydrogen bonds in the dissipative network, and the fast water migration enabled by the interconnected porous structures, described herein below. Prompt stress relaxation is beneficial for cell proliferation and migration; it can also help regulate the fate of stem cells.

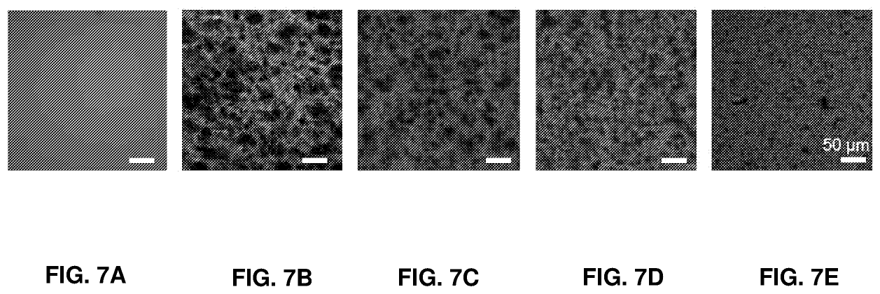

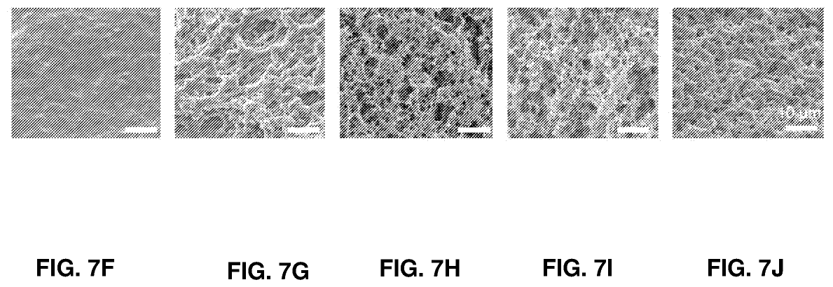

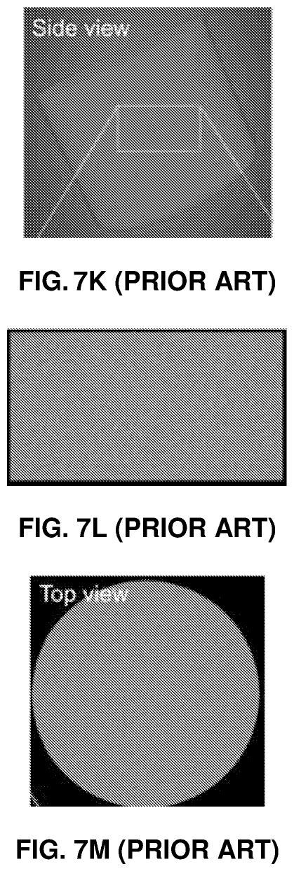

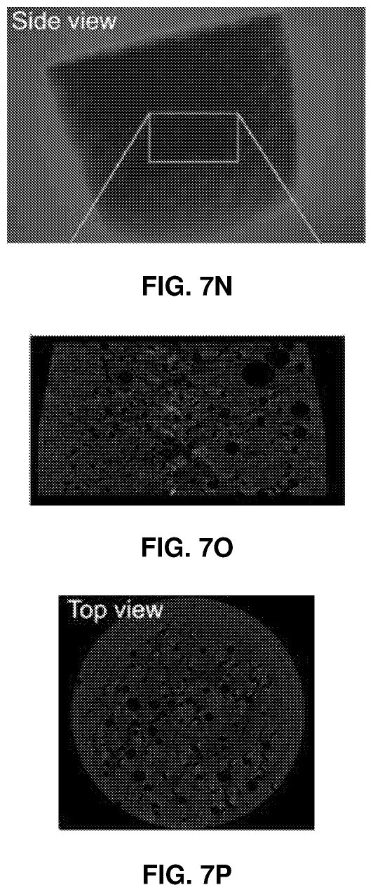

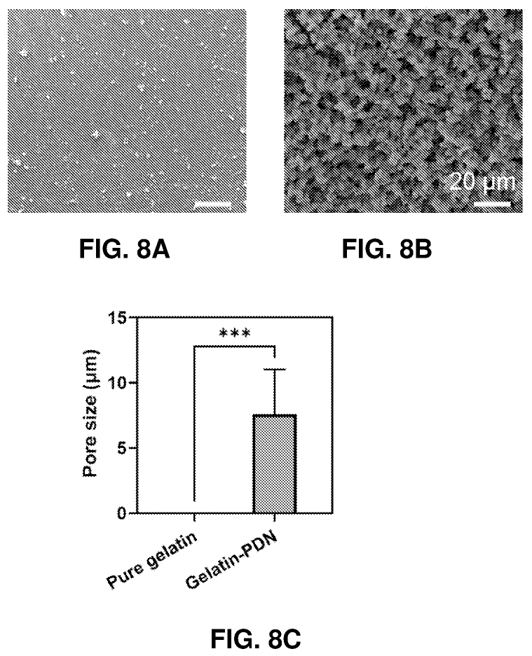

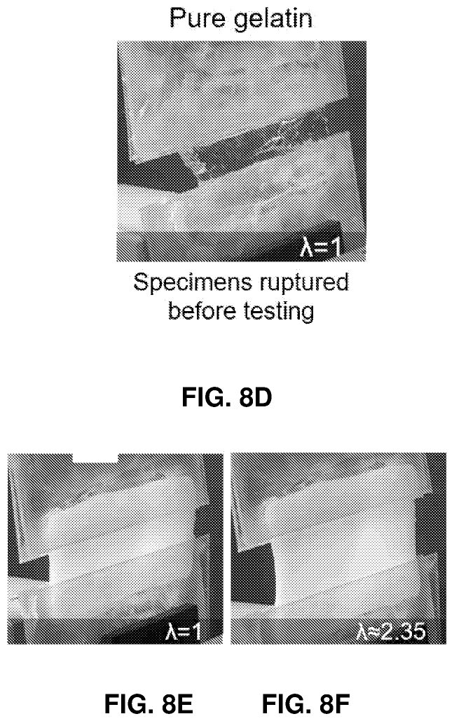

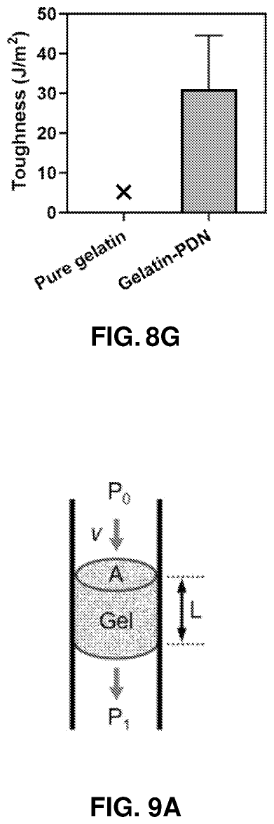

A salient feature of PDNs is their interconnected microporous structure. To characterize the structural properties, the three types of hydrogels (NSN, PSN, and PDN) were synthesized and visualized containing FITC-labeled macromolecules with a confocal microscope at wet state. This process involved no drying or lyophilization treatment. NSN displayed no detectable pores. The mesh size of NSN and most existing injectable hydrogels was on the order of 10 nm, therefore well below the resolution limit of the confocal microscope (FIGS. 1E-1J). In contrast, PSN and all PDNs displayed micrometer-sized pores resulting from the phase separation of chitosan, which was further confirmed by the fluorescence intensity distribution. A single peak was observed for NSN, indicating a homogenous network. PSN and PDNs displayed a wide intensity distribution that included areas with low or even no fluorescence. The porous structures was also verified using SEM and μCT (FIGS. 7A-7P). For SEM, the samples were dehydrated with a CO₂ supercritical dryer to minimize artifacts. For μCT, the samples were scanned in hydrated condition. Both techniques confirmed the presence of an interconnected porous structure within PDNs, concluding the pore-forming capacity of our hydrogels (FIGS. 8A-8F).

Both the pore size and porosity were tunable by adjusting the concentration of the secondary network polymer—glycol-chitosan. The average pore size varied between 6 to 10 μm, comparable to the size of cells (FIG. 1K). The porosity can be tuned over a range of ˜21-54% (FIG. 1L). The concentration of glycol-chitosan is inversely proportional to the average pore size and porosity. This relationship was attributed in part to the interplay between the phase separation and the crosslinking of glycol-chitosan. With increasing glycol-chitosan concentration, the crosslinking of glycol-chitosan accelerated, and thus reduced the mobility of the chitosan and the time window for the phase separation. As a consequence, the proportion of the polymer-poor phase, i.e., pores and porosity, is decreased. The results further underscore the importance of orchestrating the gelation and phase separation processes for desired porous structures.

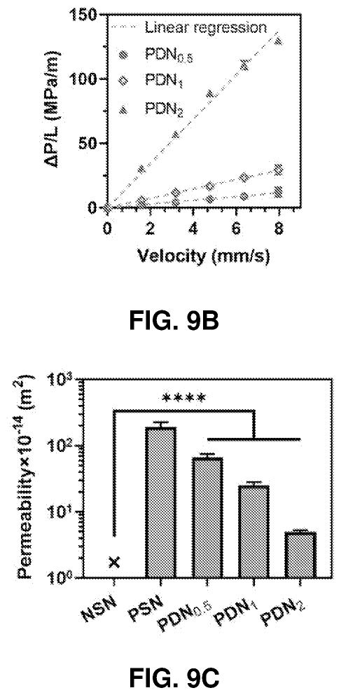

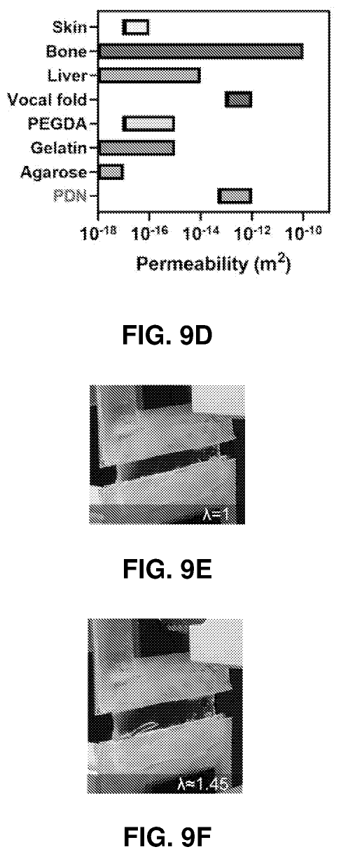

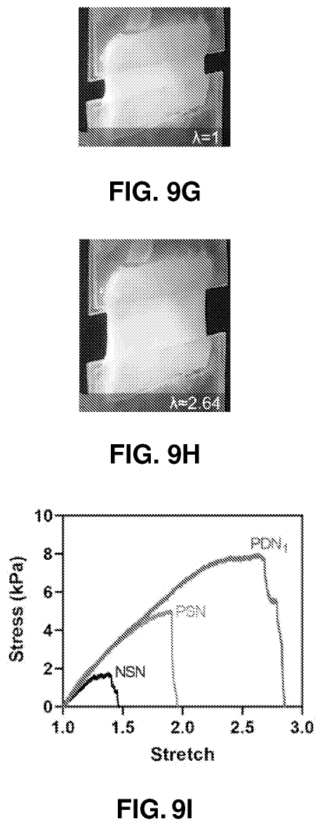

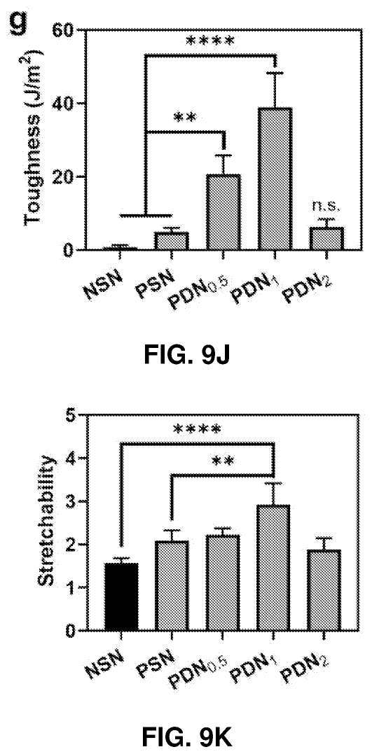

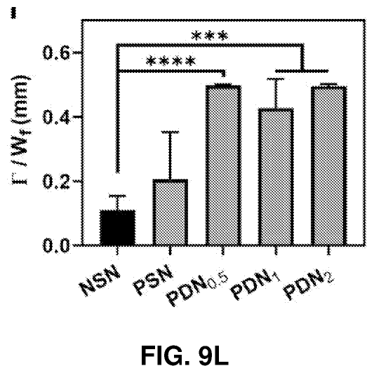

The interconnected porous structures of PDNs enabled superior permeability compared to NSN and PSN. Permeability governs fluid transport within a hydrogel and mass exchange with the surrounding environment. High permeability supports the survival, activities, and function of cells in deep layers of hydrogels by ensuring adequate nutrient and oxygen delivery. This is especially important when immediate vascularization is lacking. To characterize the permeability, k, of PDNs, cylindrical hydrogel samples were perfused with media at various flow rates Q, while measuring the pressure drop, ΔP=P₀−P₁, using a pressure transducer (FIG. 9A and FIGS. 10A-10C). Following Darcy's law, the following was calculated: k=μLQ/AΔP, where μ is the dynamic viscosity of the media (8.90×10⁻⁴ Pa·s for water), and A and L are the cross-sectional area and thickness of the hydrogel, respectively. The normalized pressure drop (ΔP/L) across the PDN sample was linearly proportional to flow velocity, confirming an ideal porous material flow resistivity behavior (FIG. 9B) which means that the porous structure was stable under different hydraulic pressures. This behavior is preferred as it indicates that the porous structure of the hydrogel is stable and does not collapse easily. The permeability of PDNs was on the order of 10⁻¹⁴ to 10⁻¹² m² (FIG. 9C). In contrast, it was not possible to perfuse media through NSN without fracturing the hydrogel due to its low permeability. The permeability results were also compared with values for commonly used hydrogels and biological tissues (FIG. 9D). PDNs exhibited at least 2 to 4 orders of magnitude greater permeability than most existing hydrogels. The measured permeability demonstrated that the PDNs contain highly interconnected porous structure, enabling rapid convection of transport fluid within the matrix.

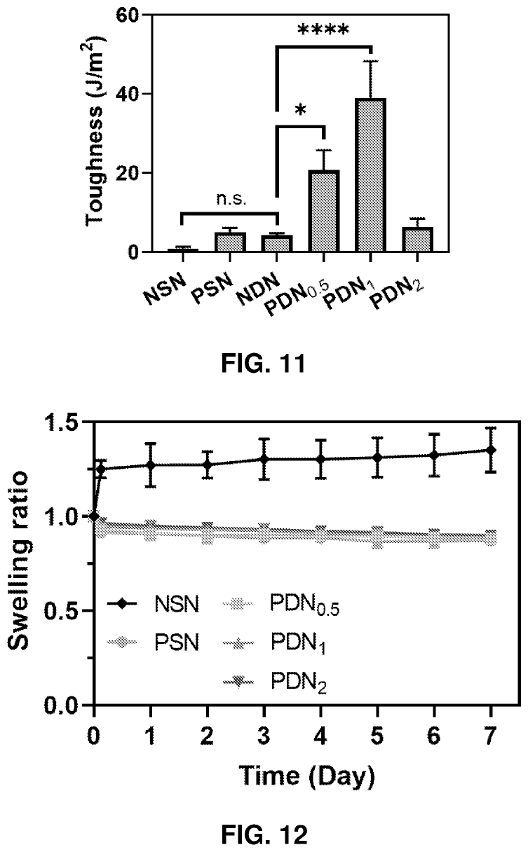

Despite the highly porous structure, PDNs are mechanically tough and insensitive to pores. The toughness was measured with pure shear tests (FIGS. 9E-9H). The area under the stress-stretch curve before a critical stretch, which was measured with a notched specimen, is the critical energy release rate to drive crack propagation (FIG. 9I). The toughness of NSN and PSN was ˜1 and 5 J m⁻², respectively. These values are in accordance with past reports on the toughness of single-network hydrogels (around 1 to 10 J m⁻²). In comparison, PDN_{0.5} and PDN₁ exhibited a fracture toughness of 20 and 39 J m⁻², respectively, corresponding to 20- and 40-fold increases compared to NSN (FIG. 9J). The stretchability of PDN₁ was also twice higher than that of NSN (FIG. 9K). The enhanced toughness and stretchability of PDNs can be attributed to the double-network configuration. Without wishing to be bound by theory, the physical crosslinks of chitosan break to dissipate energy under strain, while the covalent crosslinks of glycol-chitosan retain structural integrity. Notably, the toughening performance from the phase separation of chitosan was more significant compared to commonly used dissipative network, such as calcium crosslinked alginate. A nanoporous double-network hydrogel (NDN) was prepared by replacing chitosan with calcium-alginate within PDN. Although NDN improved the toughness by 5-fold compared to NSN, the achieved toughness was still one order of magnitude lower than that of PDN₁ (FIG. 11). To evaluate the pore-sensitivity of the hydrogels, the fractocohesive lengths were compared (characteristic crack lengths below which a material is insensitive to its presence). Unlike single-network hydrogels, for which pores act as defects, the PDNs of the present disclosure demonstrated a fractocohesive length of up to 0.5 mm (FIG. 9L). Owing to this high flaw-insensitive threshold, the pores within the PDNs did not degrade their toughness or act as defects. A detailed summary of toughness, permeability, pore size, porosity, and stress relaxation data is shown in Table 4 below.

TABLE 4
Summary of structural and mechanical properties of representative hydrogels and
biological tissues.

| | | Pore | Porosity | Toughness | Permeability | T_{1/2} | Cytocompatible | Reference |
|---|---|---|---|---|---|---|---|---|
| | | size (μm) | (%) | Γ(m⁻²) | (m²) | (S) | synthesis? | |
| Injectable | PDN of the | 6-10 | ~21-54 | Γ: 5-39 | 10⁻¹⁴-10⁻¹² | 10¹-10² | Yes | |
| tough | present | | | W^{a}: ~14 | | | | |
| hydrogels | disclosure | | | λ^{b}: ~3 | | | | |
| | MethGH-HA | N/A^{c} | ~0 | Γ: n/a | N/A | N/A | Yes | Rodell et |
| | hydrogel | | | W: 9-14 | | | | al. 2016 |
| | | | | λ: ~3 | | | | |
| | Dual-click tough | N/A | ~0 | N/A | N/A | N/A | Yes | Truong et |
| | hydrogel | | | | | | | al. 2015 |
| | PVA-CPBA/Ca | N/A | ~0 | N/A | N/A | N/A | Yes | Zhao et al. |
| | | | | | | | | 2018 |
| | PVA-Bioglass | N/A | N/A | N/A | N/A | N/A | Yes | Zhao et al. |
| | | | | | | | | 2019 |
| | Fibrin-gelatin | ~10 | ~35 | Γ: n/a | N/A | N/A | Yes | Mu etal. |
| | nanoparticles | | | W: 9-10 | | | | 2020 |
| | | | | λ: ~1.5 | | | | |
| Commonly | Alginate | 0.005-0.017 | ~0 | 1-10 | N/A | 10²-10⁴ | Yes | Sun et al. |
| used | | | | | | | | 2012; |
| hydrogels | | | | | | | | Chaudhuri |
| | | | | | | | | et al. |
| | | | | | | | | 2016; |
| | | | | | | | | Augst et |
| | | | | | | | | al. 2016 |
| | Agarose | 0.08-0.4 | N/A | 15 | 10⁻¹⁷-10⁻¹⁶ | 10²-10³ | Yes | Kwon et |
| | | | | | | | | al. 2011; |
| | | | | | | | | Johnson |
| | | | | | | | | et al. |
| | | | | | | | | 1996; |
| | | | | | | | | Righetti et |
| | | | | | | | | al. 1981 |
| | Chitosan | <0.1 | ~0 | 1-10 | N/A | 10⁴ | Yes | Bao et al. |
| | | | | | | | | 2020 |
| | Gelatin | 0.012-0.03 | N/A | 0.5-5 | 10⁻¹⁸-10⁻¹⁵ | 10³-10⁴ | Yes | Thakre et |
| | | | | | | | | al. 2018; |
| | | | | | | | | Miller et al. |
| | | | | | | | | 1951; |
| | | | | | | | | Koshy et |
| | | | | | | | | al. 2016; |
| | | | | | | | | Mi ri et al. |
| | | | | | | | | 2018 |
| | Hyaluronic acid | 0.005-0.012 | N/A | N/A | N/A | 10²-10⁴ | Yes | Xu et al. |
| | | | | | | | | 2012; Lou |
| | | | | | | | | et al. 2018 |
| | PEGDA | 0.007-0.025 | ~0 | N/A | 10⁻¹⁷-10⁻¹⁵ | N/A | Yes | Offeddu et |
| | | | | | | | | al. 2018; |
| | | | | | | | | Chiu et al. |
| | | | | | | | | 2011 |
| | Polyacrylamide | ~0.01 | ~0 | 10-500 | 10⁻¹⁸-10⁻¹⁶ | >10⁴ | No | Sun et al. |
| | | | | | | | | 2012; |
| | | | | | | | | Kapur et |
| | | | | | | | | al. 1996 |
| | Collagen gel | 1.1-2.2 | N/A | N/A | 10⁻¹⁶-10⁻¹⁵ | 10⁰-10² | Yes | Ramanujan |
| | | | | | | | | et al. |
| | | | | | | | | 2002 |
| Preformed | Bioprinted | 18-53 | ~10-50 | N/A | N/A | N/A | Yes | Ying et al. |
| porous | GelMA | | | | | | | 2020 |
| scaffolds | | | | | | | | |
| | Alginate cryogel | ~30-100 | ~70 | N/A | N/A | N/A | No | Bencherif |
| | | | | | | | | et al. 2012 |
| | Collagen sponge | 95-150 | >99 | N/A | 10⁻¹³ | N/A | No | O’Brien et |
| | (freeze-dried) | | | | | | | al. 2006 |
| | Bioglass foam | ~300 | ~90-95 | N/A | 10⁻¹⁹ | N/A | No | Ochoa et |
| | | | | | | | | al. 2009 |
| | Polycaprolactone | ~1000 | ~30-70 | N/A | 10⁻¹⁰-10⁻⁸ | N/A | No | Mitsak et |
| | scaffold | | | | | | | al. 2011; |
| | | | | | | | | Dias et al. |
| | | | | | | | | 2012 |
| Biological | Vocal fold | ~1-100 | ~90 | 160-450 | 10⁻¹³-10⁻¹² | ~60 | Not applicable | Xu etal. |
| tissues | | | | | | | | 2008, Miri |
| | | | | | | | | et al. 2016 |
| | Liver | 0.1 | ~20 | 160 | 10⁻¹⁸-10⁻¹⁴ | 500 | | Chaudhuri |
| | | | | | | | | et al. |
| | | | | | | | | 2016, |
| | | | | | | | | Gokgol et |
| | | | | | | | | al. 2012, |
| | | | | | | | | Raghunathan |
| | | | | | | | | et al. |
| | | | | | | | | 2010 |
| | Skin | 5-500 | ~80 | 1000-20000 | 10⁻¹⁷-10⁻¹⁶ | N/A | | Zhang et |
| | | | | | | | | al. 2016, |
| | | | | | | | | Holyoke et |
| | | | | | | | | al. 1952, |
| | | | | | | | | Oftadeh et |
| | | | | | | | | al. 2018 |
| | Tendon | 4-12 | ~60-70 | N/A | 10⁻²¹-10⁻¹⁷ | ~1 | | Oftadeh et |
| | | | | | | | | al. 2018, |
| | | | | | | | | Ramakrishna |
| | | | | | | | | et al. |
| | | | | | | | | 2019, |
| | | | | | | | | Butler et |
| | | | | | | | | al. 1997 |
| | Intervertebral | 0.0015 | N/A | N/A | AF: 10⁻¹⁷ | ~1 | | Cortes et |
| | disc | | | | NP: 10⁻¹⁸ | (NP) | | al. 2013 |
| | Bone | 6-300 | ~3-80 | 400-30000 | 10⁻²⁵-10⁻¹⁰ | N/A | | Cardoso |
| | | | | | | | | et al. |
| | | | | | | | | 2013, |
| | | | | | | | | Renders |
| | | | | | | | | et al. 2007 |
| | Small-intestinal | 1-10 | ~87 | N/A | 10⁻¹⁷ | N/A | | Renders |
| | submucosa | | | | | | | et al. |
| | | | | | | | | 2007, |
| | | | | | | | | Beatty et |
| | | | | | | | | al. 2002 |
| | Articular | ~0.006 | ~75 | 690-1300 | 10⁻¹⁷ | 1500 | | Ma et al. |
| | cartilage | | | | | | | 2021, |
| | | | | | | | | DiDomenico |
| | | | | | | | | et al. |
| | | | | | | | | 2018 |

^{a)}W: work of fracture, kJ m⁻³.
^{b)}λ: Stretchability.
^{c)}N/A: not available.

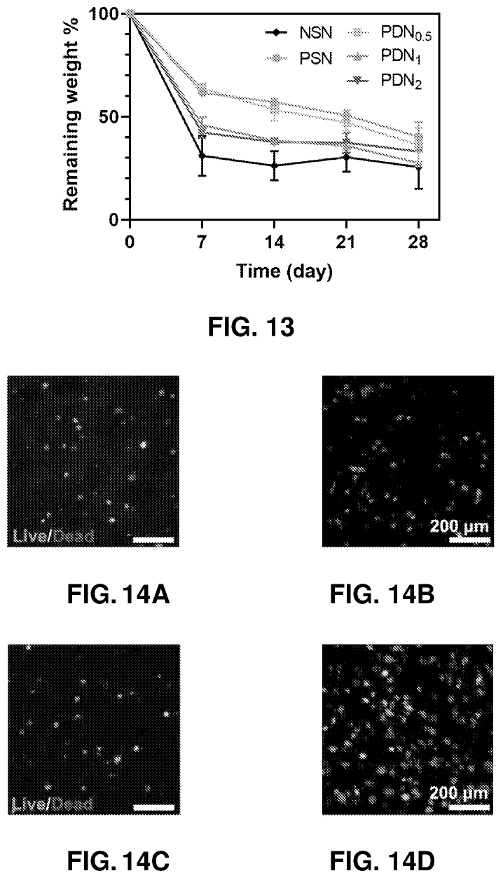

As swelling affects the mechanical and physical robustness, the swelling profile of PDNs was evaluated next. Due to difficulties in accurately measuring the weight of hydrated porous materials, the swelling was quantified by monitoring the dimensional change of PDNs upon immersion in phosphate-buffered saline (PBS). PDNs maintained their original sizes with less than 10% size change while swelling greater than 30% was observed in NSN over a 7-day period (FIG. 12). Good physical stability in a liquid environment helps hydrogels maintain their shape, which is important in ensuring that surrounding tissues are not subjected to undue compressive stresses. PDNs are also biodegradable by enzymes. They showed a slow degradation profile over 28 days when exposed to lysozyme at the physiological level (FIG. 13). Such a degradation rate is helpful in supporting the growth of encapsulated cells while they secrete their own matrix to form new tissue.

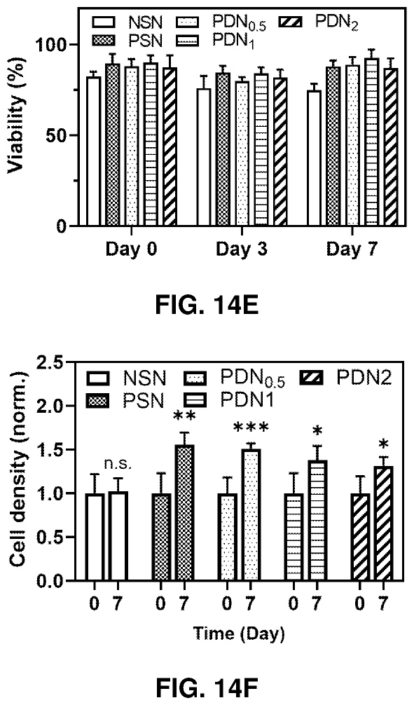

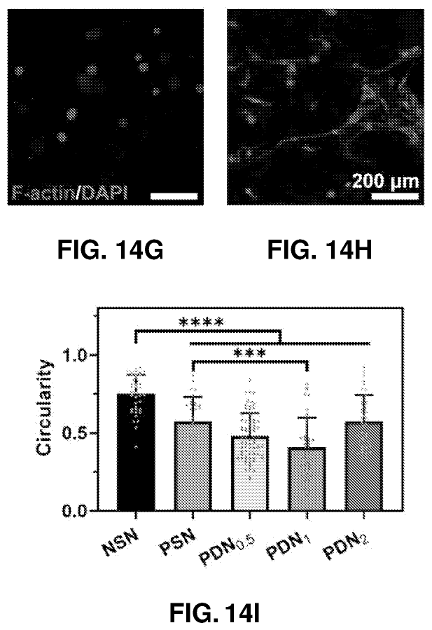

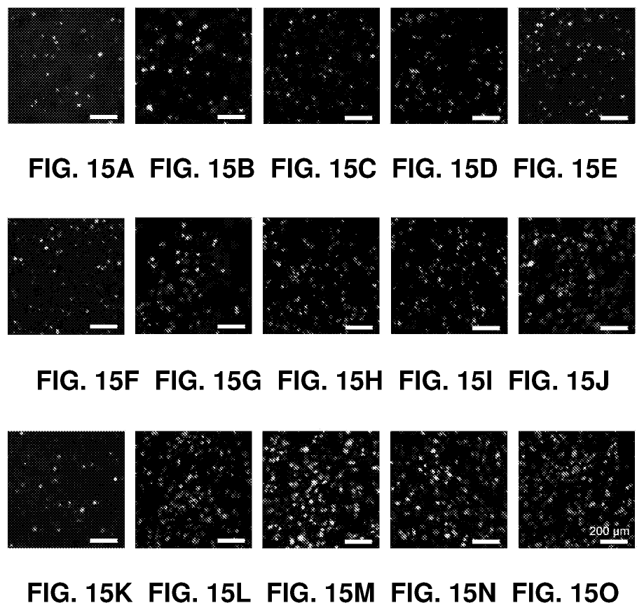

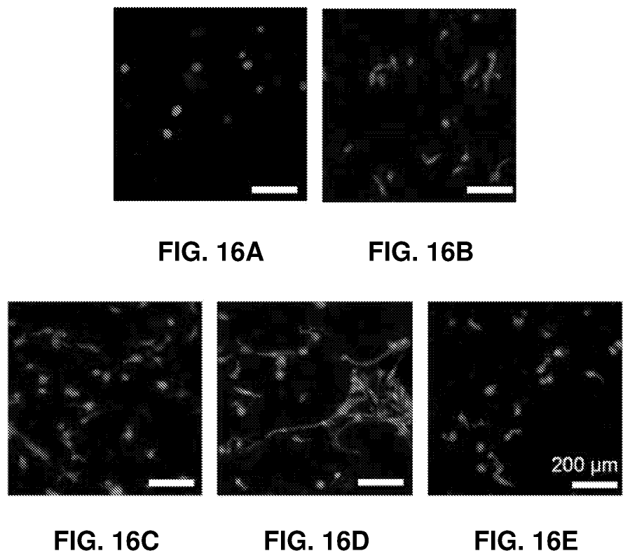

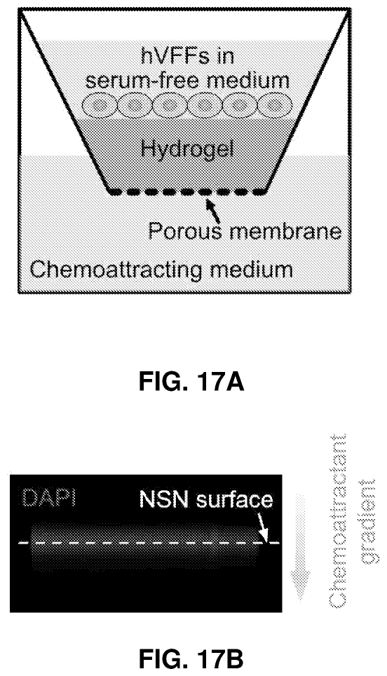

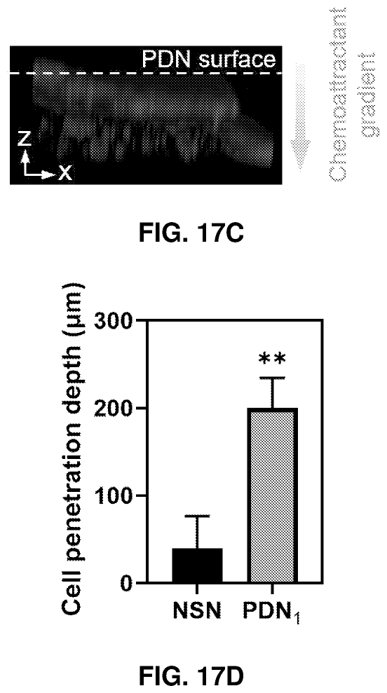

Injectable hydrogels for cell encapsulation and delivery must be cytocompatible and supportive of cell growth. These biological characteristics of PDNs were evaluated with human vocal fold fibroblasts (hVFFs), one of the main cell types found in mechanically dynamic vocal fold tissues. The cells were encapsulated within the hydrogels during a 7-day culture. LIVE/DEAD assays showed that all the NSN, PSN, and PDNs are cytocompatible. The cell viability for PDNs exceeded 85% in all cases and was consistently higher than that of NSN (FIGS. 14A-14E, and 15A-15O). Hydrogels used here were not fluorescently labeled and thus not visualized. Substantial hVFFs proliferation further confirmed that PDNs provided a cell-friendly 3D environment (FIG. 14F). In contrast, cells cannot proliferate in NSN, likely due to the nanoporous matrix imposing excessive mechanical constraints that restricted cellular activities. A similar conclusion was drawn from assessments of cell morphology. The hVFFs elongated within the 3D porous matrices of PDNs, while those cultured in NSN maintained spherical shapes (FIGS. 14G-14H and 16A-16E). FIG. 14I shows a substantial difference in cell circularity between the nanoporous and porous gels, supporting the importance of porous structure in promoting cell spreading. Cells were also found to penetrate into PDN₁ matrix within a 2-day culture period under a chemoattractant gradient but not NSN (FIGS. 17A-17D). The results demonstrated the function of pores in facilitating cell recruitment and migration. Considering its excellent mechanical, structural, and biological properties, PDN₁ was chosen for the subsequent investigations, unless otherwise specified.

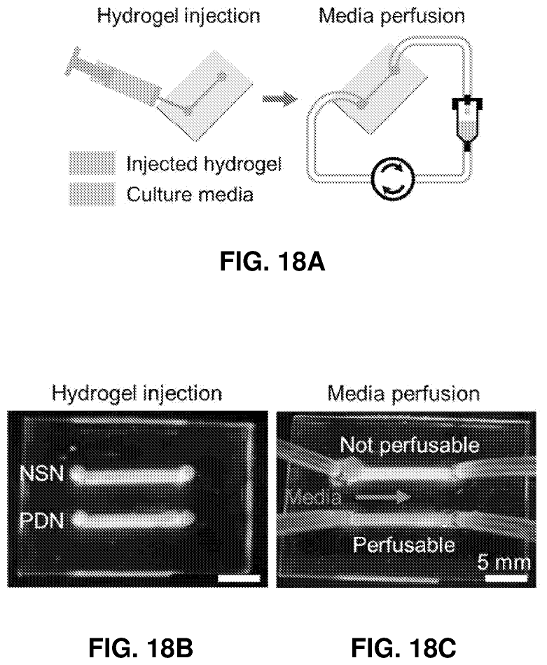

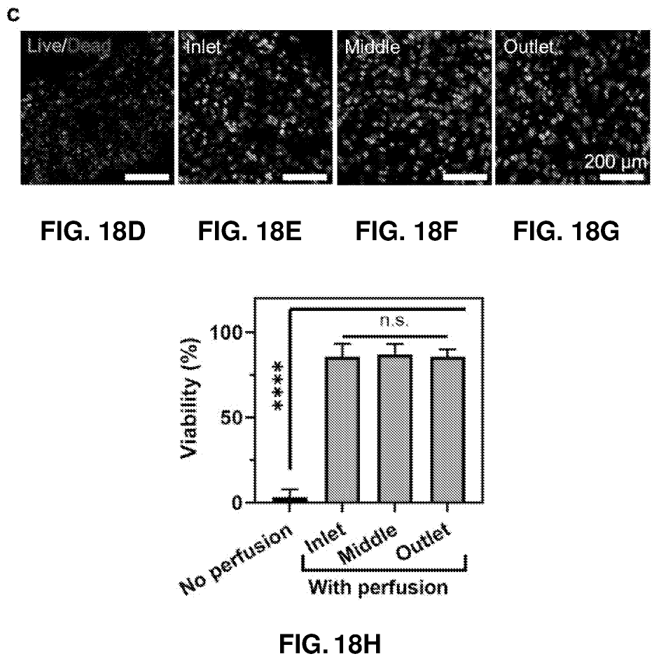

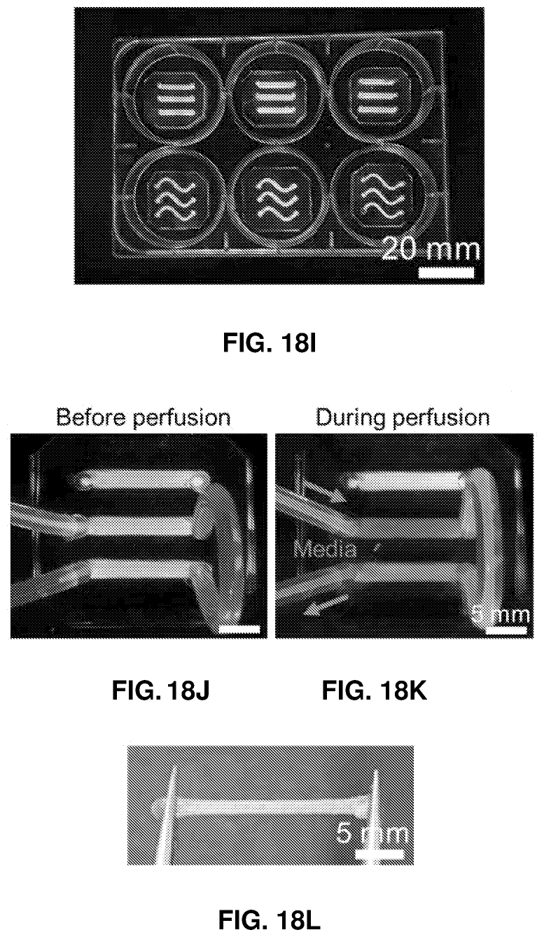

The use of PDN was first explored in a miniaturized perfusable 3D culture device, where PDN was injected into a microfluidic channel (FIG. 18A). The injectability of the PDN eliminates the need for high-precision prefabrication of tight-fitting inserts for microfluidic devices. To visualize the function of the device, FITC-labeled hydrogels (green) were prepared and added rhodamine dyes into PBS to simulate perfusion media (red). FIGS. 18B-18C show the direct media perfusion through the PDN matrix, thanks to its interconnected porous structures and high permeability. No channel blockage or media leakage was observed. By examining the distribution of the fluorescent signals, it was confirmed that media perfused through the entire channel. In contrast, NSN blocked the microfluidic channels and prevented media flow.

To demonstrate the cell culture with PDN in microfluidics, hVFFs were cultured in the PDN-laden devices. The hVFFs were mixed into the hydrogel precursors and injected into the device's channels. The cell-laden device was then perfused with cell culture media for 24 hours. A standard culture condition without perfusion was included as control. Viability assays confirmed excellent cell viability throughout the PDN channel, including the inlet, the middle, and the outlet (FIGS. 18D-18H). In contrast, most cells were dead in the control due to a lack of oxygen and nutrients. The setup was also readily useable in well plates, similar to those used in microfluidic devices (FIG. 18I). Notably, the high permeability and low flow resistance of PDNs allow the perfusion of multiple channels connected in series with a single syringe input (FIGS. 18J-18K). This enables a modular design for the co-culture of multiple cells in different compartments. Owing to their mechanical toughness, the injected PDNs can be easily harvested and manipulated after their maturation (FIG. 18L). They can be sectioned to perform multiple assays in parallel, such as different immunochemistry staining tasks. The advantages of PDN thus include simplifying the design and operation of 3D cell culture microfluidics.

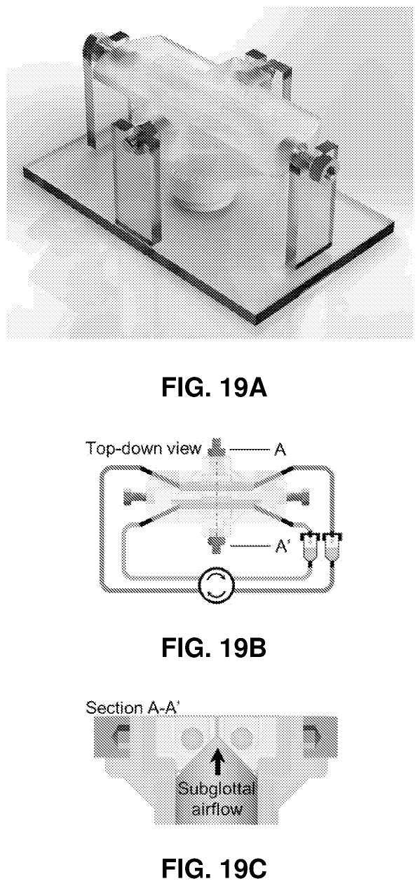

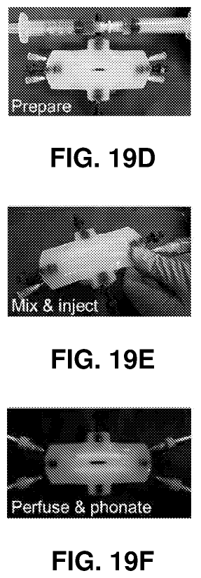

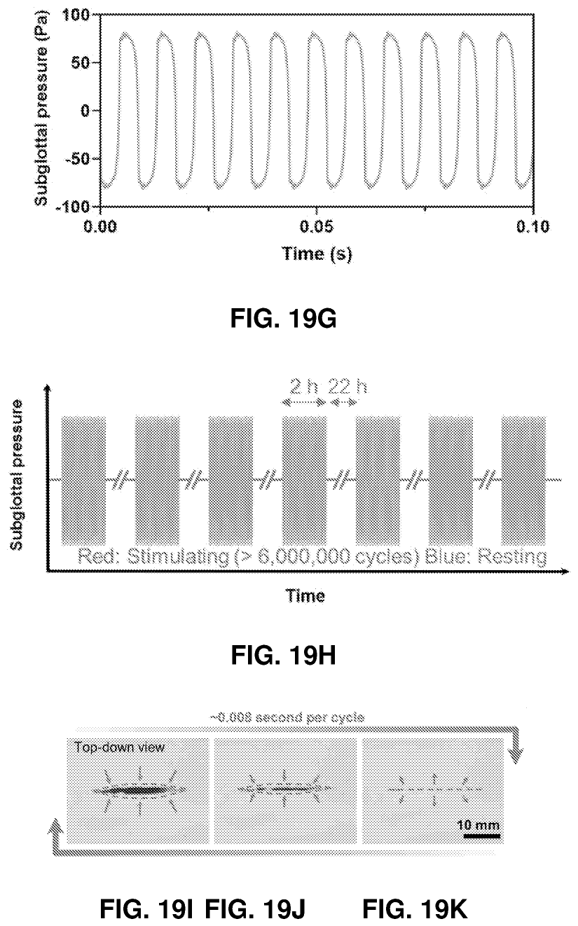

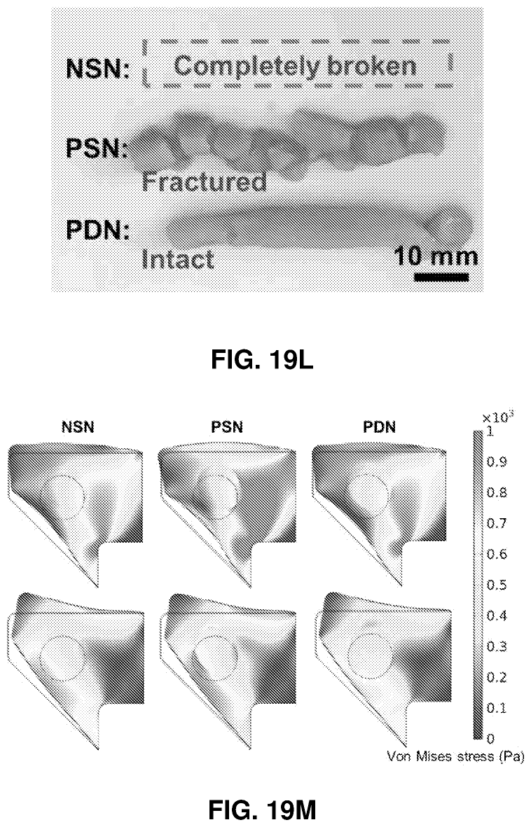

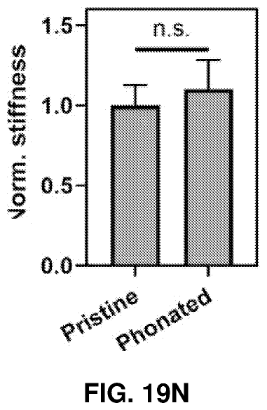

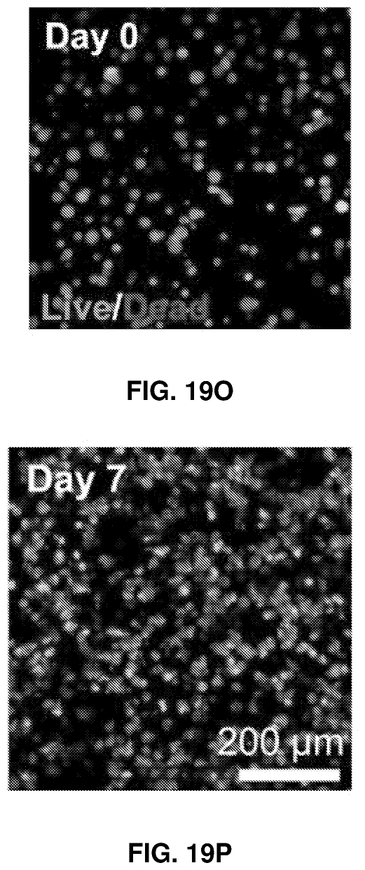

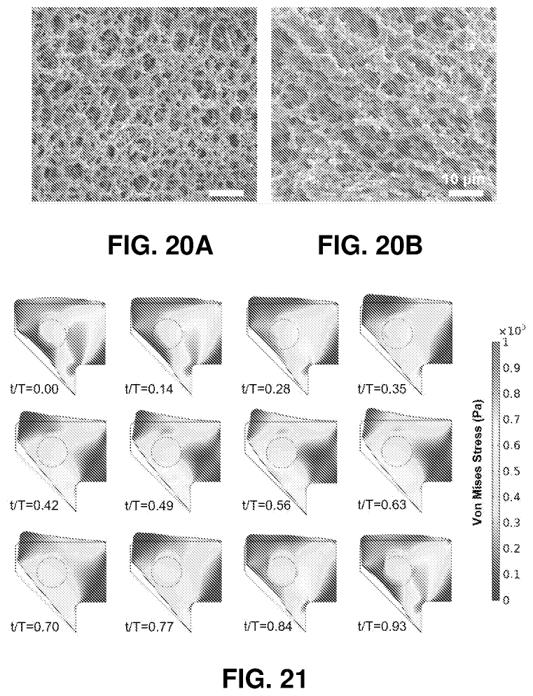

To test the resilience of PDN under complex physiological conditions, a phonomimetic perfusable bioreactor was used to simulate biomechanical stimulations of the vocal folds (FIGS. 2A-2C). FIGS. 19A-19C illustrate the structure of the bioreactor, which contains a pair of elastomer-based vocal fold bodies covered by a thin outer layer, representing the lamina propria and the epithelium of the real tissue. Each vocal fold body contained a cavity where the PDN can be easily injected, perfused, and be subjected to phonation stresses thereafter. Medium was perfused through hypodermic needles inserted through the elastomer to reach the cavity and ensure a hermetic seal (FIGS. 19D-19F). Phonation was achieved with controlled airflow across the subglottal area that induced self-oscillation of the vocal fold bodies (FIG. 19C, section A-A′). The phonation frequency and subglottal pressure were controlled to within physiologically relevant range (FIG. 19G). In particular, the frequency was kept at ˜120 Hz, similar to the fundamental frequency of humans when voicing. The injected hydrogels were phonated for 2 hours/day for 7 days, inducing a total of over 6,000,000 cycles of vibrations (FIG. 19H). The lips of the bioreactor close, collide, and open during each phonation cycle that lasts 0.008 s (FIGS. 19I-19). After the completion of biomimetic stimulations, the hydrogels were harvested from the bioreactor and it was found that PDN withstood the extreme biomechanical environment and maintained their integrity (FIG. 19L). The porous structure was also found similar to the pristine state after cyclic loading under perfusion (FIGS. 20A-20B). In contrast, NSN disintegrated into small particles that were washed away by the perfusion media, and PSN fractured into multiple disjoint chunks.

To further reveal the mechanical environment in the bioreactor, finite element analysis was conducted to probe the mechanical loading applied onto the hydrogels (FIG. 19M and FIG. 21). Both elastomers and hydrogels were treated as hyperelastic materials with the Ogden model and conjugated with damping. The PDN model showed the lowest maximum von Mises stress and the most homogenous stress distribution among the three conditions, due to its excellent energy dissipation under stress. It was also verified experimentally that the stiffness of PDN was not affected by the cyclic mechanical stimulations (FIG. 19N). Despite the complex loadings, PDN was found to support cellular viability and proliferation for the encapsulated cells throughout the 7-day culture period (FIGS. 19O and 19P). Cell culture media were able to penetrate the entire 6 cm-thick scaffold thanks to its exceptional permeability. Accordingly, it was surprisingly found that the present material (i.e. PDN supported cell viability in a centimeter-scale avascular construct.

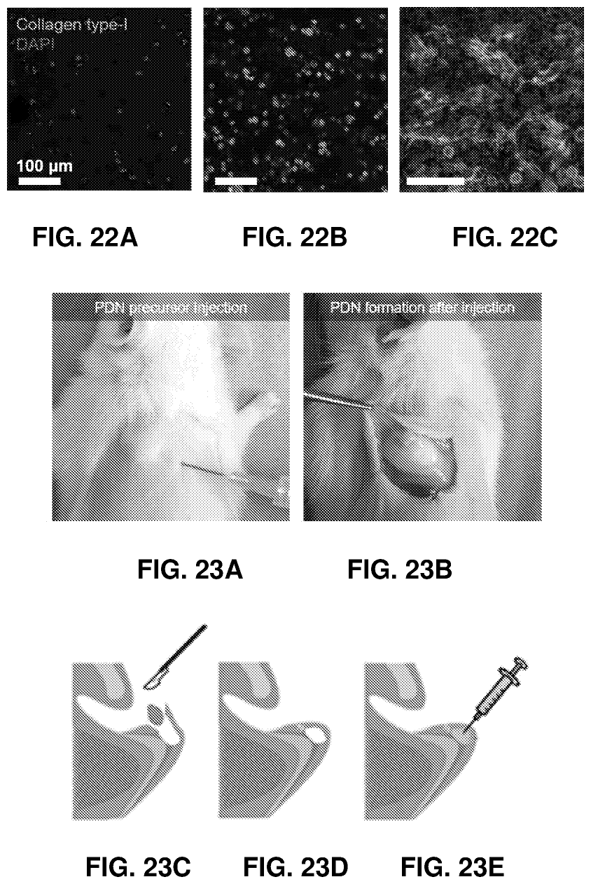

PDN also showed great translational potential. It was found that encapsulated hVFFs secreted more collagen content under dynamic stimulations compared to cells that cultured statically, indicating the stability of PDN could help activate encapsulated cells to produce a functionalized tissue (FIGS. 22A-22C). In addition, it was demonstrated herein that PDNs can be injected into animals subcutaneously to form a porous hydrogel in situ without leakage (FIGS. 23A-23B). The present results demonstrate that PDNs can be used to repair mechanically active tissues such as vocal folds after lesion removal (FIG. 23C-23E).

It was then investigated whether the second polymer network can be crosslinked enzymatically, to improve the biocompatibility compared to chemical crosslinking methods. To this end, the self-crosslinked chitosan was maintained as the primary network but an enzyme was used to crosslink bioactive gelatin to form the secondary network. Gelatin is a protein acquired from the denaturation of collagen, which is the major protein constituent of many vital tissues in the body. Gelatin has low immunogenicity and avoids the pathogen transmission risk associated with collagen. Gelatin can be crosslinked by a naturally occurring protein crosslinking enzyme, such as transglutaminase (TG), and form a stable hydrogel at body temperature. TG is an innocuous enzyme widely used in the food industry-approved for human intake by the U.S. Food and Drug Administration (FDA). TG functions by catalyzing the reaction between ε-amino groups of lysine residues and γ-carboxyamide groups of glutaminyl residues, resulting in the formation of intramolecular and intermolecular ε-(γ-glutaminyl)-lysine isopeptide bonds and formation of a permanent network of polypeptides. TG maintains its high-level activity over a broad range of temperatures (37-50° C.) and pH values (˜90% between pH=5 to 8) which makes it compliant with a wide variety of biomaterial fabrication techniques.

To fabricate enzymatically crosslinked PDNs, different concentrations of polymer precursor and gelling agent were prepared separately: a polymer precursor that contains 2.5% chitosan and 1.25-5% gelatin in 0.2 M acetic acid solution at 37° C.; and a relevant gelling agent comprising SC (0.445 M) and TG (50-250 mg/mL). Once mixing is initiated, the SC in the gelling agent elevates the pH of the mixture from acidic to physiological and induces the chitosan phase separation to form the dissipative network, following the procedures described in the previous sections. At the same time, TG enzymatically crosslinks the gelatin to create the covalently crosslinked stretchy network. The process is chemical-free and cell-friendly. Cell suspension can be added to the mixture before injection. The enzymatically crosslinked PDNs are referred to herein as PDN_{x,y}, where x and y represent the w/v percentage of gelatin content and the TG/gelatin ratio degree (L denotes low and H denotes high enzyme concentrations), respectively. The final concentration of chitosan for all the PDNs was set at 1.5%. Nanoporous single-network (NSN) hydrogels made of 5% TG-crosslinked pure gelatin. Porous single-network (PSN) hydrogels made up of 1.5% pure chitosan were used as controls.

The materials used for enzymatically crosslinked PDNs are described as follows: Chitosan (DDA: 95%, medium and high molecular weight) was purchased from Xi'an Lyphar Biotech. Chitosan powder (2.5%) was added to 0.2 M acetic acid and rotated overnight to obtain a homogeneous solution. Various concentrations of gelatin (G2500) were dissolved in the chitosan solution at 37° C. to prepare PDN precursors. To prepare the gelling agent, a phosphate solution (PS) was prepared by mixing 0.1 M sodium phosphate dibasic (Na₂HPO₄, S7907) and 0.1 M sodium phosphate monobasic (NaH₂PO₄, S8282) with a volume ratio of 50:3. Sodium bicarbonate (SC, S233-500, Fisher Scientific™) was then added to the phosphate solution, and the pH was adjusted with 1 M HCl to 9.5. The gelling solutions were then completed by adding different concentrations of microbial transglutaminase (TG, Activa-TI™, Ajinomoto North America Inc., Il., U.S.) into the phosphate buffer solution. Hydrogels of various compositions were formed by mixing precursor solutions and their associated gelling agent at a 3:2 volume ratio and using a syringe connector. The exact ingredients for each formulation are listed in Table 5.

TABLE 5
Formula for synthesizing enzymatically crosslinked PDNs

| | Hydrogel precursor | Gelling agent |
|---|---|---|
| NSN | 7.50% gelatin in PBS | 25% TG in PBS |
| PSN | 2.5% chitosan in 0.2M acetic acid | 0.445M SC in PS |
| PDN_{1.25, L} | 1.25% gelatin + 2.5% | 0.445M SC + 5% |
| | chitosan in 0.2M acetic acid | TG in PS |
| PDN_{1.25, H} | 1.25% gelatin + 2.5% | 0.445M SC + 10% |
| | chitosan in 0.2M acetic acid | TG in PS |
| PDN_{2.5, L} | 2.50% gelatin + 2.5% | 0.445M SC + 10% |
| | chitosan in 0.2M acetic acid | TG in PS |
| PDN_{2.5, H} | 2.50% gelatin + 2.5% | 0.445M SC + 15% |
| | chitosan in 0.2M acetic acid | TG in PS |
| PDN_{3.75, L} | 3.75% gelatin + 2.5% | 0.445M SC + 15% |
| | chitosan in 0.2M acetic acid | TG in PS |
| PDN_{3.75, H} | 5.00% gelatin + 2.5% | 0.445M SC + 20% |
| | chitosan in 0.2M acetic acid | TG in PS |
| PDN_{5, H} | 3.75% gelatin + 2.5% | 0.445M SC + 20% |
| | chitosan in 0.2M acetic acid | TG in PS |
| PDN_{5, H} | 5.00% gelatin + 2.5% | 0.445M SC + 25% |
| | chitosan in 0.2M acetic acid | TG in PS |

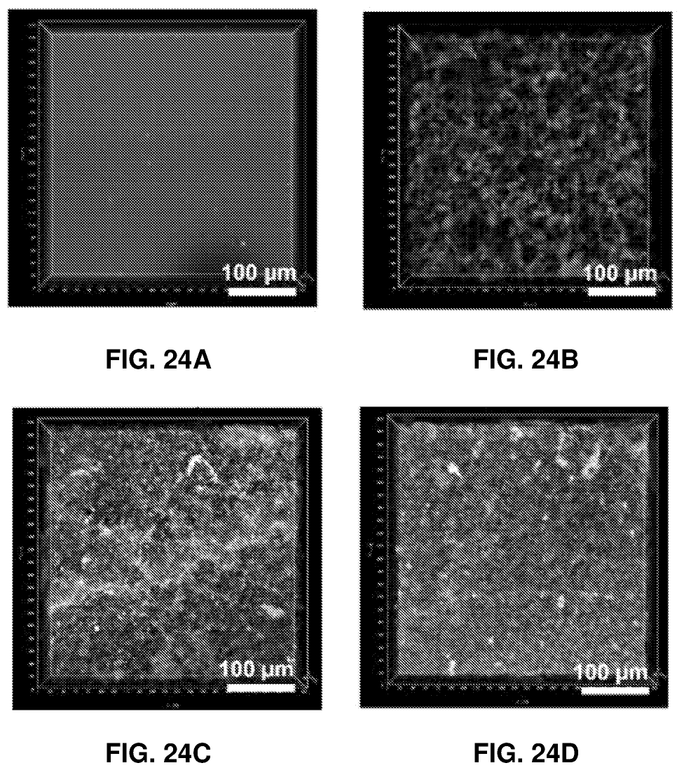

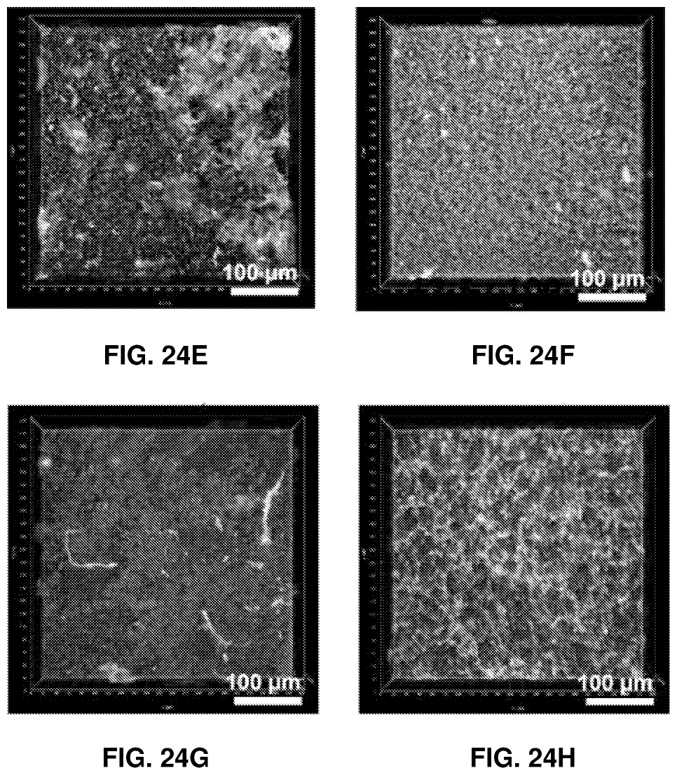

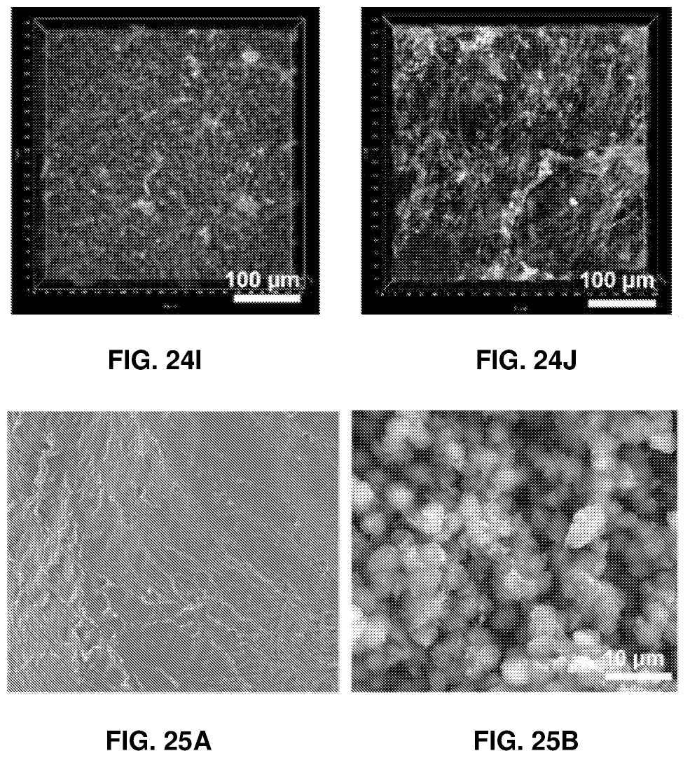

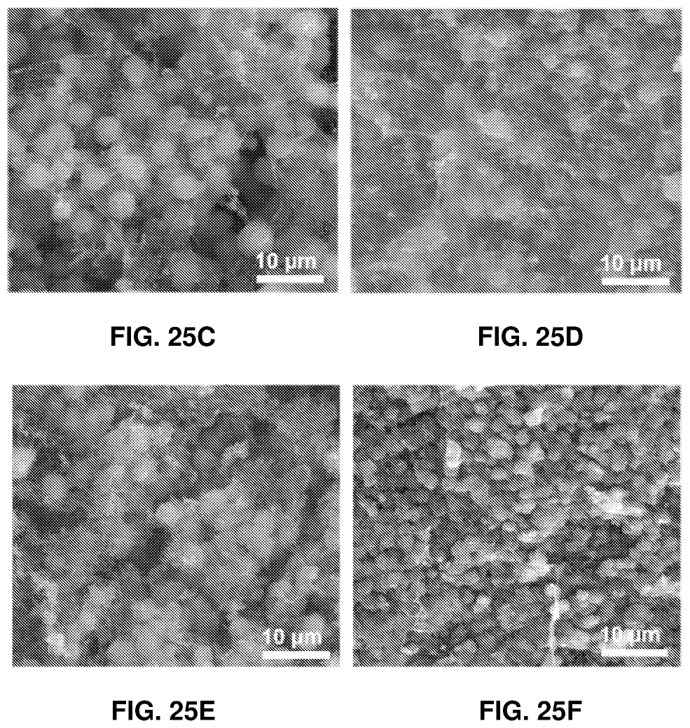

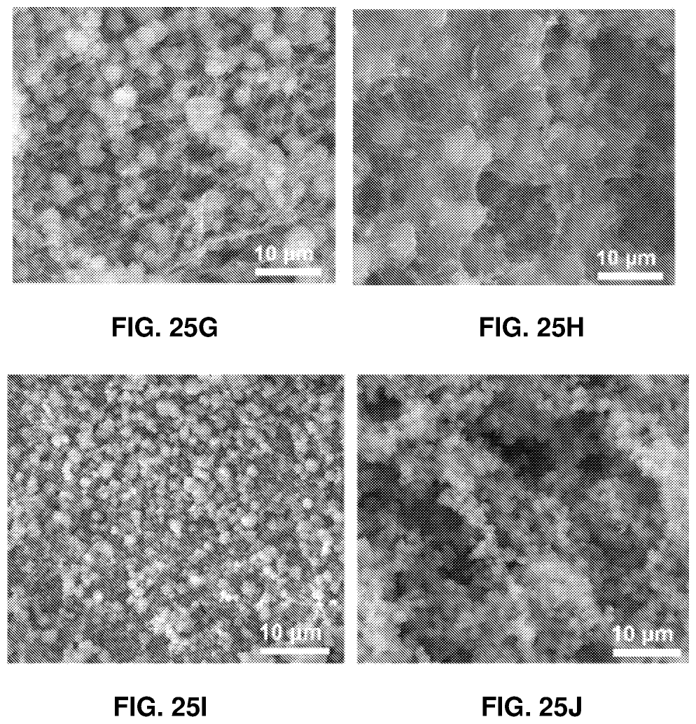

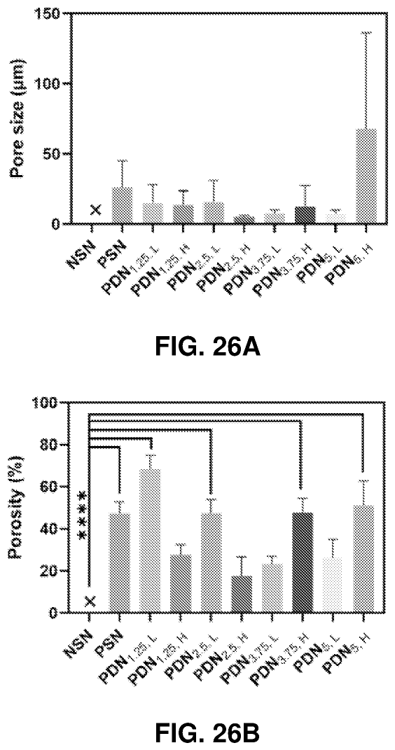

The pore-formation with enzymatically crosslinked PDN was confirmed using confocal microscopy (FIGS. 24A-24J) and SEM (FIGS. 25A-25J). Green fluorescent FITC and red fluorescent Rhodamine B was labeled to gelatin and chitosan, respectively, to reveal the distribution of the two polymer networks. It was found that interconnected pores formed with the enzymatic crosslinking method, while single-network gelatin did not form any visible pores. Both the pore size and porosity are tunable by adjusting the concentration of the secondary network polymer (gelatin) and its enzymatic crosslinker (TG). The average pore size varied between 6 to 65 μm, which is comparable to the size of cells (FIGS. 26A-26B). The porosity can be tuned over a range of ˜21-71%. The pore size and porosity are inversely proportional to the concentrations of gelatin (up to 2.5%) and TG (up to 250 mg/mL). When the concentrations of gelatin and/or TG are at a low level, the gelation of the secondary network happens at a much slower rate than the phase separation of chitosan. As a result, the gelatin chains have enough time to diffuse from the polymer-rich phase, formed by the phase separation of chitosan, to the polymer-rich phase, thereby diminishing the porous structure. On the contrary, when gelatin or TG concentration increases, gelatin can be immobilized quickly within the polymer-rich phase and promote the formation of large pores. Notably, the largest pore size and porosity were achieved in samples with the highest concentration of gelatin (5%) and TG (250 mg/mL). This relationship was attributed to the interplay between the rate of phase separation and the covalent crosslinking kinetics. The results further highlight the importance of orchestrating the gelation and phase separation processes for desired porous structures.

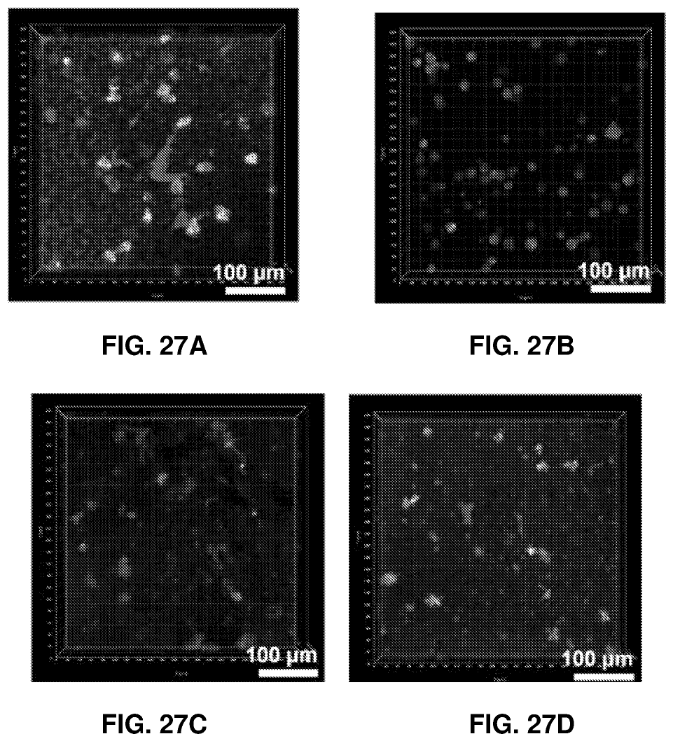

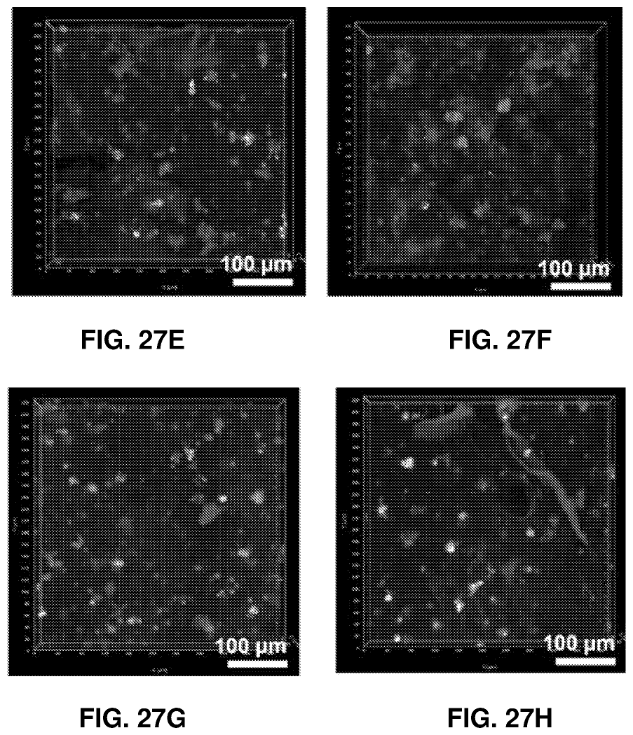

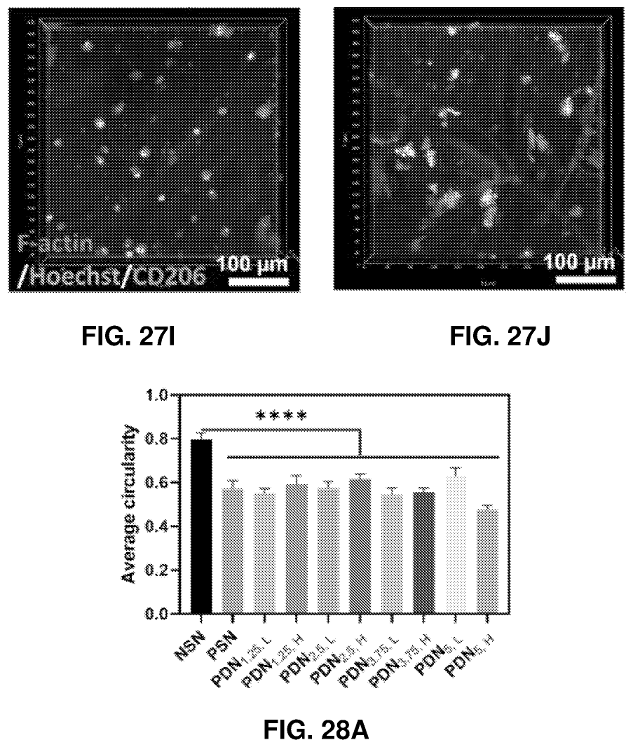

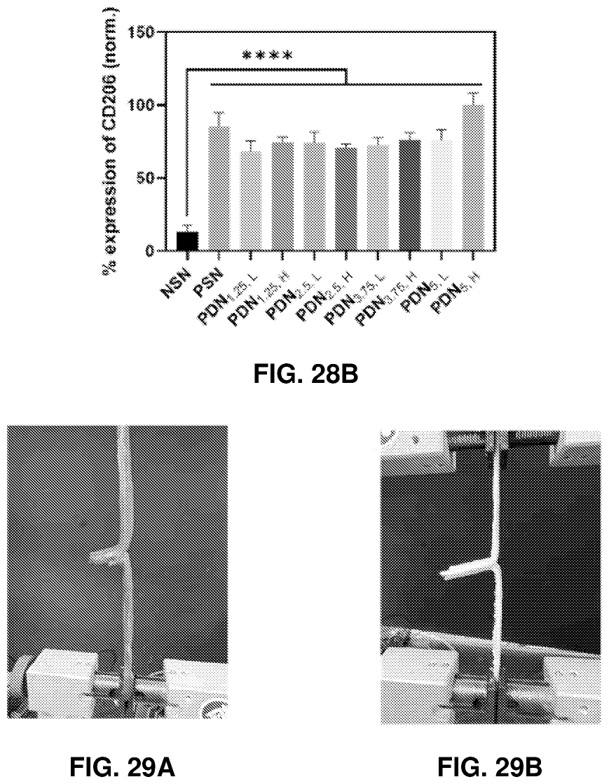

Immune cells such as macrophages play an important role in wound healing process. The PDNs' capability to control macrophage spreading and polarization was assessed in vitro. Immunofluorescence staining was used to compare the morphology and polarization of macrophages after six days of encapsulation within different hydrogels. The signal of the M2 macrophages in the pro-healing state (M2 for short) characteristic CD206 was also quantified by an object-based 3D-surface segmentation method using Imaris™ software. As shown in FIGS. 27A-27J, macrophages encapsulated within the NSN presented a round pancake-like morphology, while the cells encapsulated within the PSN and PDNs looked flatter and more irregular. Based on the graph shown in FIG. 28A, the degree of cell elongation was higher when encapsulated within the hydrogels with a larger average pore size. A similar result was obtained from the expression amount of CD206 that was most intense in PSN and PDNs (FIG. 28B). These findings suggest a strong correlation between the pore size of PDN and macrophage elongation and M2 phenotype polarization. In scaffolds with large pores, macrophages orient themselves more easily, develop a more natural and spread-out morphology, and acquire the tissue reparative roles performed by the M2s. The present data corroborates the understanding that macrophage elongation promotes polarization toward a pro-healing M2 phenotype.

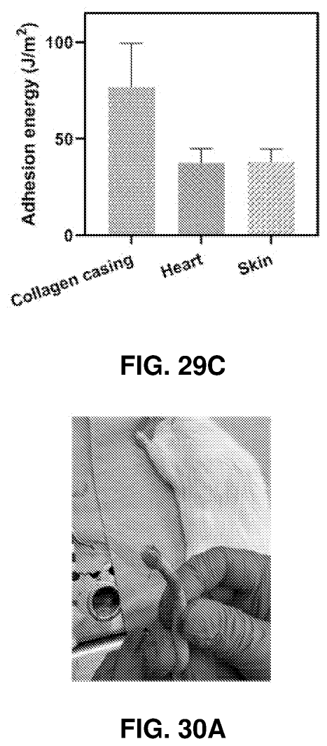

The retention of hydrogels to the injured sites are critical to ensure wound healing efficacy. Traditional methods such as sutures, staples, and mechanical fasteners allow immediate closure of the wound but are often accompanied by high localized stress, thereby causing further tissue damage and scarring at the wound site. Alternative strategies for non-invasive wound closure and wound healing simplify surgical procedures and enhance treatment efficiency by reducing the risk of adverse effects. In particular, tissue adhesives offer advantages such as lower stress concentration by eliminating mechanical mismatch, ease of implementation, improved cosmetic outcome, and localized delivery of therapeutic agents. Therefore, the adhesive properties of PDN were characterized on various substrates, including collagen casing, porcine heart, and skin tissues. Peeling PDN from the collagen casing yielded adhesion energy of ˜76 J m⁻² (FIGS. 29A-29C). High adhesion performance was also achieved across the biological tissues (30-47 J m⁻²), exceeding the values of fibrin glues and medical tapes (10-20 J m⁻²). Strong tissue adhesion using PDN could be attributed to both coupling reagent and bridging polymers. TG offers a chemical-free way to couple the tissues and the PDN, both rich in the primary amine and glutamine groups. Moreover, the highly motile chitosan network within the PDN matrix acts as a bridging polymer that migrates and penetrates the hydrogel-tissue interface. This mechanism assures the reliability of the adhesion between the PDN and the substrate.

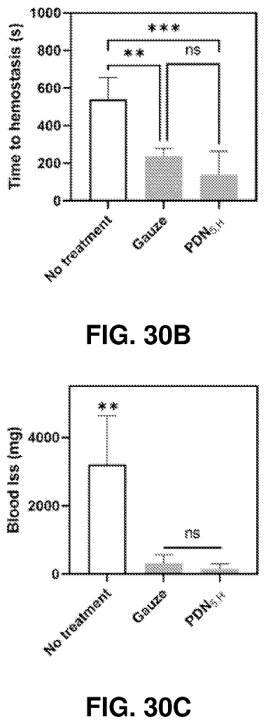

Injectable hydrogels that can simultaneously stop the bleeding and promote wound healing are beneficial for surgeries or trauma injuries. An ideal injectable hemostatic hydrogel should exhibit rapid gelation to immediately and reliably control bleeding. A rat tail amputation hemorrhage model was used to evaluate the blood clotting potential of PDNs (FIG. 30A). FIG. 30B shows that the injury with no treatment took 500 seconds to achieve hemostasis, similar to the normal human blood clotting time reported in the literature (430 s). The positive control was a generic laparotomy sponge (Gauze). When PDN hydrogel was used to treat the injured site, the time for hemostasis obtained was reduced to 93±3 s. The mass of blood loss was also less when the PDN (478±8 mg) was applied compared to the no-treatment group (3230±850 mg) (FIG. 30C). The decreased blood hemostasis time and mass of blood loss for the hydrogel is due to the hemostatic sealing and bioadhesion ability of PDN. Chitosan has inherent hemostatic ability. Charged amino groups on chitosan interact with negatively charged proteins and glycolipids on the surface of red blood cells resulting in the adhesion and aggregation of blood cells and thus endowing the enzymatically crosslinked PDN with effective blood-clotting capability. Moreover, enzymatically crosslinked PDN is highly porous and presents hemostatic agents, such as chitosan and TG, providing a large contact area for the interaction with the blood components and accelerating the clotting cascade. The synergy of adhesion, open pores, and coagulation promotion capabilities of enzymatically crosslinked PDNs shows great potential to treat various hemorrhage situations.

### REFERENCES

Y. Alsaid, et al. Adv. Mater. 2021, 33, 2008235.
A. D. Augst, et al. Macromol. Biosci. 2006, 6, 623
G. Bao, et al. Mater. Horiz. 2020, 7, 2336.
M. W. Beatty, et al. Tissue Eng. 2002, 8, 955.
S. A. Bencherif, et al. PNAS 2012, 109, 19590.
S. L. Butler, et al. Biomed. Eng. 1997, 5.
L. Cardoso, et al. J. Biomech. 2013, 13.
O. Chaudhuri, et al. Nature Mater. 2016, 15, 326.
Y.-C. Chiu, et al. Biomaterials 2011, 32, 6045.
D. H. Cortes, et al. J. Biomech. 2014, 47, 2088.
M. R. Dias, et al. J. Biomech. 2012, 45, 938.
C. D. DiDomenico, et al. Nat. Rev. Rheumatol. 2018, 14, 393.
C. Gokgol, et al. Med. Eng. Phys. 2012, 34, 882.
J. B. Holyoke, et al. J. Investig. Dermatol. 1952, 18, 147.
E. M. Johnson, et al. AlChE J. 1996, 42, 1220.
V. Kapur, et al. Ind. Eng. Chem. Res. 1996, 35, 3179.
S. T. Koshy, et al. Adv. Healthc. Mater. 2016, 5, 541.
H. J. Kwon, et al. Polym. Eng. Sci. 2011, 51, 1078.
J. Lou, et al. Biomaterials 2018, 154, 213.
Z. Ma, et al. Adv. Mater. 2021, 33, 2007663.
M. Miller, et al. J. Phys. Chem. 1951, 55, 1387.
A. K. Miri, et al. Acta Biomater. 2018, 77, 38.
A. K. Miri, et al. J. Voice 2016, 30, 251.
A. G. Mitsak, et al. Tissue Eng. Part A 2011, 17, 1831.
Z. Mu, et al. Adv. Healthcare Mater. 2020, 9, 1901469.
F. J. O'Brien, et al. Technol. Health Care. 2006, 15, 3.
I. Ochoa, J. A. Sanz-Herrera, et al. J. Biomech. 2009, 42, 257.
G. S. Offeddu, et al. AIP Adv. 2018, 8, 105006.
R. Oftadeh, et al. Acta Biomater. 2018, 70, 249.
S. Raghunathan, et al. Ann. Biomed. Eng. 2010, 38, 1789.
H. Ramakrishna, Biodegradable Biphasic Scaffolds using Braiding Technology and Surface Treatments for Regeneration of Tendon-Bone Junction Tissue. Doctoral thesis, North Carolina State University, Raleigh, N.C., 2019.
S. Ramanujan, et al. Biophys. J. 2002, 83, 1650.
H. Ravanbakhsh, et al. (2019). Materials Science and Engineering. 2019, C, 103, 109861.
G. A. P. Renders, et al. 2007, 210, 239.
P. G. Righetti, B et al. J. Biochem. Bioph. Meth. 1981, 4, 347.
C. B. Rodell, et al. Adv. Mater. 2016, 28, 8419.
R. C. Scherer, et al. J. Acoust. Soc. Am. 2001, 109, 1616.
J.-Y. Sun, et al. Nature 2012, 489, 133.
A. A. Thakre, et al. Sci. Technol. 2018, 32, 1899.
V. X. Truong, et al. J. Am. Chem. Soc. 2015, 137, 1618.
C. C. Xu, et al. Tissue Eng. Part A 2008, 14, 1893.
X. Xu, et al. Soft Matter 2012, 8, 3280.
G. Ying, et al. Adv. Fund. Mater. 2020, 30, 2003740.
Q. Zhang, et al. Acta Biomater. 2016, 35, 166.
X. Zhao, et al. Chem. Rev. 2021, 121, 4309.
Y. Zhao, et al. J. Mater. Chem. B 2018, 6, 1351.
Y. Zhao, et al. Adv. Healthcare Mater. 2019, 8, 1900709.

What is claimed is:

1. A porous double-network hydrogel comprising:
a first network comprising a first polymer;
a second network comprising a second polymer; and
wherein the porous double-network hydrogel comprises pores having a diameter of at least 1 μm, and wherein the porous double-network hydrogel is perfusable and injectable.

2. The porous double-network hydrogel of claim 1, wherein the weight to volume ratio of the weight of the first polymer to the volume of the hydrogel is from 0.25 to 4%.

3. The porous double-network hydrogel of claim 1, wherein the weight to volume ratio of the weight of the second polymer to the volume of the hydrogel is from 0.15 to 8%.

4. The porous double-network hydrogel of claim 1, wherein the pores have a maximal diameter of 100 μm.

5. The porous double-network hydrogel of claim 1, wherein the porous double-network hydrogel has a porosity of 18 to 70%.

6. The porous double-network hydrogel of claim 1, wherein the porous double-network hydrogel has a fracture toughness of at least 5 J m⁻².

7. The porous double-network hydrogel of claim 1, wherein the porous double-network hydrogel has a permeability of from 10⁻¹⁴ to 10⁻¹² m².

8. The porous double-network hydrogel of claim 1, wherein the porous double-network hydrogel has a half-life time of stress relaxation from 10 to 100 s.

9. The porous double-network hydrogel of claim 1, wherein the porous double-network hydrogel has a fractocohesive length of from 0.25 mm to 0.5 mm.

10. The porous double-network hydrogel of claim 1, wherein the first polymer has a pKa of from 6 to 6.5.

11. The porous double-network hydrogel of claim 1, wherein the first polymer is physically self crosslinked.

12. The porous double-network hydrogel of claim 1, wherein the first polymer is chitosan or poly(N-isopropylacrylamide).

13. The porous double-network hydrogel of claim 1, wherein the second polymer is covalently crosslinked.

14. The porous double-network hydrogel of claim 12, wherein the second polymer is self crosslinked.

15. The porous double-network hydrogel of claim 12, wherein the second polymer is enzymatically crosslinked.

16. The porous double-network hydrogel of claim 15, wherein the second polymer is enzymatically crosslinked by peptide bonds.

17. The porous double-network hydrogel of claim 1, wherein the second polymer is glycol-chitosan, polyacrylamide, collagen, fibrin, polyethylene glycol or gelatin.

18. A method of treating a hemorrhage in a subject in need thereof comprising injecting the porous double-network hydrogel of claim 1 to a hemorrhagic site in the subject.

19. A method of treating an injury in a subject in need thereof comprising injecting the porous double-network hydrogel of claim 1 to an injury site in the subject.

20. A method of administering cellular therapy to a subject in need thereof comprising injecting the porous double-network hydrogel of claim 1 and therapeutic cells in a subject in need thereof.
